(19) United States
(12) Patent Application Publication
(10) Pub. No.: US 2024/0016954 A1
(43) Pub. Date: Jan. 18, 2024

(54) COMPOSITION FOR IMPROVING THE SOLUBILITY OF POORLY SOLUBLE SUBSTANCES, COMPLEX FORMULATION AND LIQUID FORMULATION OF AN ACTIVE INGREDIENT

(71) Applicant: INDUSTRIAL TECHNOLOGY RESEARCH INSTITUTE, Hsinchu (TW)

(72) Inventors: Wen-Chia HUANG, Taichung City (TW); Yen-Jen WANG, Taichung City (TW); Felice CHENG, Zhubei City (TW); Chia-Ching CHEN, Taichung City (TW); Shao-Chan YIN, Hsinchu City (TW); Chien Lin PAN, Hsinchu City (TW); Tsan-Lin HU, Jhubei City (TW); Meng-Nan LIN, Zhubei City (TW); Kuo-Kuei HUANG, Zhubei City (TW); Maggie LU, Hsinchu County (TW); Chih-Peng LIU, Hsinchu City (TW)

(73) Assignee: INDUSTRIAL TECHNOLOGY RESEARCH INSTITUTE, Hsinchu (TW)

(21) Appl. No.: 18/471,821

(22) Filed: Sep. 21, 2023

Related U.S. Application Data
(63) Continuation-in-part of application No. 16/232,601, filed on Dec. 26, 2018, now Pat. No. 11,801,310.
(60) Provisional application No. 62/610,509, filed on Dec. 26, 2017.

(30) Foreign Application Priority Data
Dec. 21, 2018 (EP) 18215590.3

(51) Int. Cl. A61K 47/69; A61K 47/22; A61K 9/00; A61K 9/08; A61K 47/10; A61K 47/32; A61K 47/40; A61K 47/38

(52) U.S. Cl. CPC A61K 47/6951; A61K 47/22; A61K 9/0019; A61K 9/0048; A61K 9/0053; A61K 9/08; A61K 47/10; A61K 47/32; A61K 47/40; A61K 47/38; A61K 31/47

(57) ABSTRACT

A composition for improving the solubility of poorly soluble substances is provided. The composition for improving the solubility of poorly soluble substances includes 60-97% by weight of cyclodextrin and/or a derivative thereof, 0.5-4% by weight of at least one water-soluble polymer and 0.4-30% by weight of at least one water-soluble stabilizer, wherein the at least one water-soluble stabilizer includes caffeine, and wherein the poorly soluble substance is a tyrosine kinase inhibitor.

### CROSS REFERENCE TO RELATED APPLICATION

This application is a Continuation-In-Part of pending U.S. patent application Ser. No. 16/232,601, filed on Dec. 26, 2018 and entitled “COMPOSITION FOR IMPROVING THE SOLUBILITY OF POORLY SOLUBLE SUBSTANCES, USE THEREOF AND COMPLEX FORMULATION CONTAINING THEREOF”, which claims the benefit of U.S. Provisional Application No. 62/610,509, filed on Dec. 26, 2017, and based on and claims priority from EP Application Serial Number 18215590.3, filed on Dec. 21, 2018, the disclosures of which are hereby incorporated by reference herein in their entirety.

### TECHNICAL FIELD

The present disclosure is related to a composition for improving the solubility of poorly soluble substances, the use thereof, and a complex formulation containing the composition for improving the solubility of poorly soluble substances.

### BACKGROUND

Because cyclodextrin can improve the solubility of poorly soluble drugs and avoid drug degradation, it has become an important pharmaceutical excipient.

Cyclodextrin molecules are easily bonded to each other by forming intermolecular hydrogen bonds, but such bonding easily blocks the insertion of drug molecules into the cavity of cyclodextrin, thereby causing a decrease in the drug loading amount of the cyclodextrin. Moreover, if the use of cyclodextrin in medical applications is too high, it is likely to result in a potential risk of toxicity, and that limits the amount of cyclodextrin that can be used in many drug formulations.

It is currently known that the efficiency of the inclusion effect of cyclodextrin and drugs can be improved by modifying different functional groups on cyclodextrin, adding appropriate water-soluble polymers, or the like. However, many drugs are still inefficient in forming inclusion with the existing cyclodextrins, in which the molecular size of the drug and the inner ring size of the cyclodextrin are still the main factors determining the inclusion strength of the drug and cyclodextrin.

Therefore, developing a technology capable of enhancing the inclusion effects of cyclodextrin and drugs is still needed in the current field of medical applications.

### SUMMARY

The present disclosure provides a composition for improving the solubility of poorly soluble substances, comprising 60-97% by weight of cyclodextrin and/or a derivative thereof, 0.5-4% by weight of at least one water-soluble polymer and 0.4-30% by weight of at least one water-soluble stabilizer, wherein the at least one water-soluble stabilizer comprises caffeine, and wherein the poorly soluble substance is a tyrosine kinase inhibitor.

The present disclosure also provides a complex formulation, comprising at least one active ingredient and a composition for improving the solubility of poorly soluble substances, wherein the content of at least one active ingredient in the complex formulation is 0.05-20 wt %. The at least one active ingredient is a tyrosine kinase inhibitor. The composition for improving the solubility of poorly soluble substances comprises 60-97% by weight of cyclodextrin and/or a derivative thereof, 0.5-4% by weight of at least one water-soluble polymer and 0.4-30% by weight of at least one water-soluble stabilizer, wherein the at least one water-soluble stabilizer comprises caffeine.

The present disclosure further provides a liquid formulation of an active ingredient, comprising at least one active ingredient, a composition for improving the solubility of poorly soluble substances and a solvent. The at least one active ingredient is a tyrosine kinase inhibitor. The composition for improving the solubility of poorly soluble substances comprises 60-97% by weight of cyclodextrin and/or a derivative thereof, 0.5-4% by weight of at least one water-soluble polymer and 0.4-30% by weight of at least one water-soluble stabilizer, wherein the at least one water-soluble stabilizer comprises caffeine. The content of the at least one active ingredient in the liquid formulation of an active ingredient is 0.01-10% (w/v).

A detailed description is given in the following embodiments with reference to the accompanying drawings.

### BRIEF DESCRIPTION OF DRAWINGS

The patent or application file contains at least one color drawing. Copies of this patent or patent application publication with color drawing will be provided by the USPTO upon request and payment of the necessary fee.

The present invention can be more fully understood by reading the subsequent detailed description and examples with references made to the accompanying drawings, wherein:

FIG. 1A shows the effect of the content of hydroxypropyl-β-cyclodextrin in a composition on the solubility of cabozantinib in a formulation in a solubility test for cabozantinib;

FIG. 1B shows the effect of the content of hydroxypropyl methyl cellulose in a composition on the solubility of cabozantinib in a formulation in a solubility test for cabozantinib;

FIG. 1C shows the effect of the content of caffeine in the composition on a solubility of cabozantinib in a formulation in a solubility test for cabozantinib;

FIG. 2A shows the effect of the content of hydroxypropyl-γ-cyclodextrin in a composition on the solubility of axitinib in a formulation in a solubility test for axitinib;

FIG. 2B shows the effect of the content of hydroxypropyl methyl cellulose in a composition on the solubility of axitinib in a formulation in a solubility test for axitinib;

FIG. 2C shows the effect of the content of caffeine in the composition on a solubility of axitinib in a formulation in a solubility test for axitinib;

FIG. 2D shows the effect of the content of caffeine in the composition on a solubility of axitinib in a formulation in a solubility test for axitinib (only from Sample 4 to Sample 13);

FIG. 3A shows the effect of the content of hydroxypropyl-γ-cyclodextrin in a composition on the solubility of binimetinib in a formulation in a solubility test for binimetinib;

FIG. 3B shows the effect of the content of hydroxypropyl methyl cellulose in a composition on the solubility of binimetinib in a formulation in a solubility test for binimetinib;

FIG. 3C shows the effect of the content of caffeine in the composition on a solubility of binimetinib in a formulation in a solubility test for binimetinib;

FIG. 4 shows the optimal structures of HPγCD in its closed and open forms: (a) Optimal structures of closed form; (b) optimal structures of open form; (c) a graph highlights the water molecules cannot pass through the closed-form HPγCD; (d) a graph highlights the water molecules can pass through the open-form HPγCD easily;

FIG. 5 shows the most stable structure of [LE@HPγCD] inclusion complex obtained from molecular docking analysis;

FIG. 6 shows the most stable structure of GSH-[LE@HPγCD] inclusion complex obtained from molecular docking analysis. (a) Side view; (b) Primary face view;

FIG. 7 shows the most stable structure of mannitol-[LE@HPγCD] inclusion complex obtained from molecular docking analysis;

FIG. 8 shows the most stable structure of [axitinib @HPγCD] inclusion complex obtained from molecular docking analysis;

FIG. 9 shows the most stable structure of GSH-[axitinib@HPγCD] inclusion complex obtained from molecular docking analysis. (a) Side view; (b) Primary face view;

FIG. 10 shows the most stable structure of mannitol-[axitinib@HPγCD] inclusion complex obtained from molecular docking analysis. (a) Side view; (b) Primary face view;

FIG. 11 shows the possible structures of HPMC[GSH-[LE@HPγCD]] and HPMC[GSH-[axitinib@HPγCD]] complex. (a) Hydroxypropyl methyl cellulose-GSH-[LE@HPγCD]; (b) hydroxypropyl methyl cellulose-GSH-[axitinib@HPγCD];

FIG. 12A shows the 3D structure of GSH-[LE@HPγCD] inclusion complex;

FIG. 12B shows the 3D structure of GSH-[axitinib@HPγCD] inclusion complex;

FIG. 13 shows the results of a pharmacokinetic test of HC8A solution for aqueous humor (AH) and retina of rabbit eyes in one embodiment of the present disclosure;

FIG. 14 shows the time course of an experiment of an adjuvant induced chronic uveitis model (AIU model) according to one embodiment of the present disclosure;

FIG. 15 shows the photographs of eyes of the rabbit photographed on Day 4 and Day 10 in an experiment of an adjuvant induced chronic uveitis model according to one embodiment of the present disclosure in which a vehicle, 0.1% dexamethasone sodium phosphate and the composition of the present disclosure (0.17% HPC8H8OLH/TW-PD) was respectively administered to the eyes of the rabbits at a frequency of three times a day (TID);

FIG. 16A shows the scoring results for the conjunctival congestion degree in the rabbit eyes of the vehicle treated group, the 0.1% dexamethasone sodium phosphate treated group, the composition of the present disclosure (0.17% HPC8H8OLH/TW-PD) treated group and untreated group in an experiment of an adjuvant induced chronic uveitis model according to one embodiment of the present disclosure; FIG. 16B shows the scoring results for anterior chamber flare degree in the rabbit eyes of the vehicle treated group, the 0.1% dexamethasone sodium phosphate treated group, the composition of the present disclosure (0.17% HPC8H8OLH/TW-PD) treated group and untreated group in an experiment of an adjuvant induced chronic uveitis model according to one embodiment of the present disclosure; and

FIG. 16C shows the scoring results for uveitis condition in the rabbit eyes of the vehicle treated group, the 0.1% dexamethasone sodium phosphate treated group, the composition of the present disclosure (0.17% HPC8H8OLH/TW-PD) treated group and untreated group in an experiment of an adjuvant induced chronic uveitis model according to one embodiment of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure provides a composition for improving the solubility of poorly soluble substances. As used herein, “a poorly soluble substance” means any substance having solubility in water of less than about 0.01 g/mL. The above-mentioned poorly soluble substance may comprise, but is not limited to, a hydrophobic compound, for example, may be a hydrophobic drug.

The composition for improving the solubility of poorly soluble substances mentioned above may comprise, but is not limited to cyclodextrin and/or a derivative thereof, at least one water-soluble polymer and at least one water-soluble stabilizer. In the composition for improving the solubility of poorly soluble substances of the present disclosure mentioned above, the content of each component is not particularly limited, and it may be adjusted according to the content of other components, and/or may be adjusted as needed.

In the composition for improving the solubility of poorly soluble substances of the present disclosure mentioned above, the cyclodextrin and/or a derivative thereof may occupy about 40-99.5% by weight, for example may be about 40-45%, 45-50%, 50-55%, 55-60%, 60-65%, 60-65%, 65-70%, 70-75%, 75-80%, 80-85%, 85-90%, 95-99.5%, 55-99.5%, 50-75%, 60-85%, 60-99.5%, 80-99.5% by weight, but it is not limited thereto.

Examples of the foregoing cyclodextrin may comprise α-cyclodextrin, β-cyclodextrin, γ-cyclodextrin, δ-cyclodextrin or a combination thereof, but they are not limited thereto.

Moreover, examples of the foregoing derivative of cyclodextrin may comprise hydroxypropyl modified cyclodextrin, succinyl modified cyclodextrin, methyl modified cyclodextrin or a combination thereof, but they are not limited thereto. Furthermore, the hydroxypropyl modified cyclodextrin may for example, be hydroxypropyl-β-cyclodextrin (hydroxypropyl-β or hydroxypropyl-γ-cyclodextrin (hydroxypropyl-γ-CD), but it is not limited thereto.

In the composition for improving the solubility of poorly soluble substances of the present disclosure mentioned above, the at least one water-soluble polymer may occupy about 0.05-10% by weight, for example, may be about 0.05-0.1%, 0.05-0.08%, 0.06-0.1%, 0.1-0.12%, 0.15-0.25%, 0.5-1%, 1-2%, 1-3%, 2-4%, 2-5%, 2-3%, 2.5-4%, 3-3.5%, 3-5%, 5-7%, 8-10% by weight, but it is not limited thereto.

The molecular weight of the at least one water-soluble polymer mentioned above may be greater than about 2000 Dalton, but is not limited thereto, for example, about 1000-200,000 Dalton. Moreover, the at least one water-soluble polymer mentioned above may comprise, but is not limited to hydroxypropyl methyl cellulose (HPMC), hydroxypropyl cellulose, carboxymethyl cellulose (CMC), polyvinylpyrrolidone, (PVP), polyvinyl alcohol, poly(ethylene glycol)-poly(propylene glycol)-poly(ethylene glycol) (PEG-PPG-PEG (ABA)) triblock copolymer or a combination thereof, etc. In one embodiment, in the composition for improving the solubility of poorly soluble substances of the present disclosure mentioned above, the at least one water-soluble polymer mentioned above may be hydroxypropyl methyl cellulose.

In the composition for improving the solubility of poorly soluble substances of the present disclosure mentioned above, the at least one water-soluble stabilizer may occupy about 0.05-60% by weight, for example, may be about 0.05-0.1%, 0.05-0.08%, 0.6-0.1%, 0.1-0.12%, 0.15-0.25%, 0.5-1%, 1-2%, 1-3%, 2-5%, 2-3%, 3-5%, 5-7%, 8-10%, 10-15%, 15-20%, 20-25%, 25-30%, 30-35%, 35-40%, 40-45%, 40-55%, 45-50%, 50-55%, 55-60%, 0.4-30%, 0.4-25%, 0.4-20%, 0.5-30%, 0.5-25%, 0.5-20%, 1-30%, 1-25%, 1-20%, 1-15%, 1-10%, 2-30%, 2-25%, 2-20%, 3-30%, 3-25%, 3-20%, 4-30%, 4-25%, 4-20%, 4-10%, 4.5-25%, 5-20%, 6-15%, 7-25%, 8-30%, 9-30%, 10-20%, 15-25%, 20-30%, 0.4-4%, 0.5-3.5%, 0.6-3%, 0.7-2.5%, 0.8-2.0%, 0.9-1.5%, 1-1.2%, 1.2-2%, 1.5-2.5% by weight, but it is not limited thereto.

Examples of at least one water-soluble stabilizer mentioned above may comprise, but are not limited thereto, an amino acid with a polar side chain, an oligopeptide containing at least one amino acid with a polar side chain, purine, a derivative of purine or a combination thereof.

The amino acid with a polar side chain mentioned above may be any amino acid having a polar side chain, which may be a natural amino acid or a non-natural amino acid, and is not limited. For instance, examples of the amino acid with a polar side chain may comprise glycine, cysteine, glutamine, glutamic acid or histidine, but they are not limited thereto.

Furthermore, the oligopeptide containing at least one amino acid with a polar side chain mentioned above is only required to contain at least one amino acid with a polar side chain in the amino acids constituting the same, and there is no particular limitation. For example, the oligopeptide containing at least one amino acid with a polar side chain mentioned above may have only one amino acid with a polar side chain, or may have several amino acids, each of which has a polar side chain, or the oligopeptide containing at least one amino acid with a polar side chain mentioned above may also be entirely composed of amino acids, each of which has a polar side chain. Moreover, each amino acid contained by the oligopeptide containing at least one amino acid with a polar side chain mentioned above may independently be any kind of amino acid, as long as the amino acids constituting the oligopeptide containing at least one amino acid with a polar side chain. In addition, in the oligopeptide containing at least one amino acid with a polar side chain mentioned above, the position of the at least one amino acid with a polar side chain in the oligopeptide is also not particularly limited, and it can independently be anywhere in the oligopeptide. The at least one amino acid with a polar side chain in the foregoing oligopeptide may independently comprise glycine, cysteine, glutamine, glutamic acid, histidine, any combination thereof, etc., but it is not limited thereto.

In one embodiment, the foregoing oligopeptide containing at least one amino acid with a polar side chain may have about 2-8 amino acids, such as 2-3, 2-6, 2, 3, 4, 5, 6, 7, 8 amino acids, but it is not limited thereto. Moreover, examples of the foregoing oligopeptide containing at least one amino acid with a polar side chain may be listed as carnosine, glutathione (GSH), leucine-glycine-glycine (Leu-Gly-Gly), or the like, but it is not limited thereto.

Moreover, examples of the foregoing purine may comprise adenine, guanine, a combination thereof, but they are not limited thereto. The foregoing derivative of purine may comprise, but is not limited to caffeine, theobromine, isoguanine, xanthine, hypoxanthine, uric acid, any combination thereof, etc.

In one embodiment, in the composition for improving the solubility of poorly soluble substances of the present disclosure mentioned above, the at least one water-soluble stabilizer mentioned above may be, but is not limited to, the amino acid with a polar side chain, such as glycine, glutamine, glutamic acid or histidine. In another embodiment, in the composition for improving the solubility of poorly soluble substances of the present disclosure mentioned above, the at least one water-soluble stabilizer mentioned above may be, but is not limited to, the oligopeptide containing at least one amino acid with a polar side chain, such as carnosine, glutathione, leucine-glycine-glycine. In yet another embodiment, in the composition for improving the solubility of poorly soluble substances of the present disclosure mentioned above, the at least one water-soluble stabilizer mentioned above may be, but is not limited to, the derivative of purine, such as caffeine.

As needs, the content of each component in the composition for improving the solubility of poorly soluble substances of the present disclosure mentioned above may be adjusted according to the content of other components in the composition for improving the solubility of poorly soluble substances of the present disclosure mentioned above while the total content of all component will not exceed 100 wt %.

In one embodiment, in the composition for improving the solubility of poorly soluble substances of the present disclosure mentioned above, the at least one water-soluble stabilizer mentioned above is the amino acid with a polar side chain or the oligopeptide containing at least one amino acid with a polar side chain while the cyclodextrin and/or a derivative thereof mentioned above may occupy about 40-85% by weight, the at least one water-soluble polymer mentioned above may occupy about 0.5-5% by weight, and the amino acid with a polar side chain or the oligopeptide containing at least one amino acid with a polar side chain may occupy about 15-55% by weight. Furthermore, the cyclodextrin and/or a derivative thereof mentioned above may be hydroxypropyl-γ-cyclodextrin, and the at least one water-soluble polymer mentioned above may be hydroxypropyl methyl cellulose.

Moreover, in the composition for improving the solubility of poorly soluble substances of the present disclosure mentioned above, for one specific embodiment, under the premise that the at least one water-soluble stabilizer mentioned above is the amino acid with a polar side chain or the oligopeptide containing at least one amino acid with a polar side chain, and the cyclodextrin and/or a derivative thereof, the at least one water-soluble polymer and the amino acid with a polar side chain or the oligopeptide containing at least one amino acid with a polar side chain may respectively occupy about 40-85% by weight, 0.5-5% by weight and 15-55% by weight, if it is further limited to that the cyclodextrin and/or a derivative thereof may be hydroxypropyl-γ-cyclodextrin, the at least one water-soluble polymer may be hydroxypropyl methyl cellulose, and the at least one water-soluble stabilizer may be the amino acid with a polar side chain, and the amino acid with a polar side chain may comprise glutamine, glutamic acid or histidine, in this specific embodiment, the foregoing hydroxypropyl-γ-cyclodextrin may occupy about 70-85% by weight, the foregoing hydroxypropyl methyl cellulose may occupy about 0.5-3% by weight, and the foregoing amino acid with a polar side chain may occupy about 10-25% by weight.

Alternatively, in the composition for improving the solubility of poorly soluble substances of the present disclosure mentioned above, for one specific embodiment, under the premise that the at least one water-soluble stabilizer mentioned above is the amino acid with a polar side chain or the oligopeptide containing at least one amino acid with a polar side chain, and the cyclodextrin and/or a derivative thereof, the at least one water-soluble polymer and the amino acid with a polar side chain or the oligopeptide containing at least one amino acid with a polar side chain may respectively occupy about 40-85% by weight, 0.5-5% by weight, and 15-55% by weight, if it is further limited to that the cyclodextrin and/or a derivative thereof may be hydroxypropyl-γ-cyclodextrin, the at least one water-soluble polymer may be hydroxypropyl methyl cellulose, and the at least one water-soluble stabilizer is oligopeptide containing at least one amino acid with a polar side chain, and the oligopeptide containing at least one amino acid with a polar side chain may comprise carnosine, glutathione or leucine-glycine-glycine, in this specific embodiment, the foregoing hydroxypropyl-γ-cyclodextrin may occupy about 40-80% by weight, the foregoing hydroxypropyl methyl cellulose may occupy about 0.5-3% by weight, and the foregoing oligopeptide containing at least one amino acid with a polar side chain may occupy about 15-55% by weight.

In another embodiment, in the composition for improving the solubility of poorly soluble substances of the present disclosure mentioned above, the at least one water-soluble stabilizer mentioned above may be the derivative of purine, the derivative of purine may be caffeine, the cyclodextrin and/or a derivative thereof mentioned above may be hydroxypropyl-γ-cyclodextrin, and the at least one water-soluble polymer mentioned above may be hydroxypropyl methyl cellulose. In one specific embodiment of this embodiment, the foregoing hydroxypropyl-γ-cyclodextrin may occupy about 70-99.5% by weight, the foregoing hydroxypropyl methyl cellulose may occupy about 0.1-5% by weight, and the foregoing caffeine may occupy about 0.05-20% by weight.

In further another embodiment, in the composition for improving the solubility of poorly soluble substances of the present disclosure mentioned above, the at least one water-soluble stabilizer mentioned above may be the derivative of purine, the derivative of purine may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In one specific embodiment of this embodiment, the foregoing hydroxypropyl-β-cyclodextrin or hydroxypropyl-γ-cyclodextrin may occupy about 60-97% by weight, the foregoing hydroxypropyl methyl cellulose may occupy about 0.5-4% by weight, and the foregoing caffeine may occupy about 0.4-30% by weight.

In one specific embodiment, for the composition for improving the solubility of poorly soluble substances of the present disclosure mentioned above, the poorly soluble substance may be a tyrosine kinase inhibitor, but it is not limited thereof.

Tyrosine kinase inhibitor can be categorized into seven types based on their mechanisms of action and modes of target binding. Type I tyrosine kinase inhibitors (such as cabozantinib, pazopanib) bind to the catalytic site of a target kinase in an active conformation, whereby the conserved Asp-Phe-Gly (DFG) motif of the activation loop is oriented towards the interior of the kinase and aligned with the ATP-binding site (the DFG-in conformation). Type II tyrosine kinase inhibitors (such as axitinib, regorafenib) bind reversibly to the target kinase in an inactive conformation, whereby the DFG motif is directed away from the ATP-binding site (the DFG-out conformation). Type III tyrosine kinase inhibitors (such as binimetinib, cobimetinib) bind remotely from the catalytic ATP-binding site whereby they modulate kinase activity in an allosteric manner. Type IV tyrosine kinase inhibitors (such as everolimus, sirolimus) or substrate-directed inhibitors are allosteric inhibitors that target regions outside the ATP-binding sites but do not overlap with type III inhibitors. Type V tyrosine kinase inhibitors are bivalent inhibitors that bind irreversibly to the active kinase sites and peptide motifs representing the substrate targeted by the kinase. Type VI tyrosine kinase inhibitors (such as afatinib, dacomitinib) bind covalently to their kinase target via the interaction of the reactive electrophilic groups of the inhibitors with primarily the nucleophilic cysteines. Type VII tyrosine kinase inhibitors are defined as nonclassical allosteric inhibitors that target the extracellular domain of a receptor tyrosine kinase. (The FEBS Journal 290 (2023) 2845-2864, 2022 Federation of European Biochemical Societies).

In one embodiment, for the composition for improving the solubility of poorly soluble substances of the present disclosure mentioned above, the tyrosine kinase mentioned above may comprise, but is not limited to, a Type I tyrosine kinase inhibitor, a Type II tyrosine kinase inhibitor, a Type III tyrosine kinase inhibitor, a Type IV tyrosine kinase inhibitor, a Type V tyrosine kinase inhibitor, etc., or a combination thereof.

Moreover, the Type I tyrosine kinase inhibitor mentioned above may comprise cabozantinib, pazopanib, etc., or a combination thereof, but it is not limited thereto. Type II tyrosine kinase inhibitor mentioned above may comprise, axitinib, regorafenib etc., or a combination thereof, but is not limited thereto. The Type III tyrosine kinase inhibitor mentioned above may comprise, binimetinib, etc., but is not limited thereto.

For the foregoing specific embodiment, in the composition for improving the solubility of poorly soluble substances of the present disclosure, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In the composition for improving the solubility of poorly soluble substances of the present disclosure, the foregoing hydroxypropyl-β-cyclodextrin or hydroxypropyl-γ-cyclodextrin may occupy about 60-97% by weight, such as about 60-96.5%, 65-95%, 70-90%, 75-85% by weight, but it is not limited thereto. In the composition for improving the solubility of poorly soluble substances of the present disclosure, the foregoing hydroxypropyl methyl cellulose may occupy about 0.5-4% by weight, such as about 0.55-4%, 0.6-3.95%, 0.7-3.9%, 0.8-3.85%, 0.9-3.8%, 1-4%, 1.1-3.9%, 1.2-3.8%, 1.5-3.6%, 1.8-3.5%, 2.0-3.4%, 2.2-3.2%, 2.4-3.0%, 2.6-2.8%, 2-4%, 2.5-3.5% by weight, but it is not limited thereto. In the composition for improving the solubility of poorly soluble substances of the present disclosure, the foregoing caffeine may occupy about 0.4-30% by weight, such as about 0.4-25%, 0.4-20%, 0.5-30%, 0.5-25%, 0.5-20%, 1-30%, 1-25%, 1-20%, 1-15%, 1-10%, 2-30%, 2-25%, 2-20%, 3-30%, 3-25%, 3-20%, 4-30%, 4-25%, 4-20%, 4-10%, 4.5-25%, 5-20%, 6-15%, 7-25%, 8-30%, 9-30%, 10-20%, 15-25%, 20-30%, 0.4-4%, 0.5-3.5%, 0.6-3%, 0.7-2.5%, 0.8-2.0%, 0.9-1.5%, 1-1.2%, 1.2-2%, 1.5-2.5% by weight , but it is not limited thereto.

In one embodiment, for the composition for improving the solubility of poorly soluble substances of the present disclosure, the poorly soluble substance may be a Type I tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, in the composition for improving the solubility of poorly soluble substances of the present disclosure, the foregoing hydroxypropyl-β-cyclodextrin or hydroxypropyl-γ-cyclodextrin may occupy about 65-97% by weight, such as about 66-96.9%, 70-96.5%, 75-95%, 80-92% by weight, but it is not limited thereto, the foregoing hydroxypropyl methyl cellulose may occupy about 1-4% by weight, such as about 1.1-4%, 1.2-3.9%, 1.3-3.7%, 1.5-3.5%, 2.1-3.6%, 2.5-3.5%, 2.7-3.5%, 2.8-3.0%, 2-4% by weight, but it is not limited thereto, and the foregoing caffeine may occupy about 0.7-29.5% by weight, such as about 0.75-29%, 0.8-28.5%, 3-25%, 5-22%, 8-20%, 10-18%, 12-16% by weight , but it is not limited thereto.

In one embodiment, for the composition for improving the solubility of poorly soluble substances of the present disclosure, the poorly soluble substance may be a Type II tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, in the composition for improving the solubility of poorly soluble substances of the present disclosure, the foregoing hydroxypropyl-β-cyclodextrin or hydroxypropyl-γ-cyclodextrin may occupy about 74-96% by weight, such as about 75-95%, 77-93%, 80-92%, 82-90%, 83-88% by weight, but it is not limited thereto, the foregoing hydroxypropyl methyl cellulose may occupy about 1-4% by weight, such as about 1.1-4%, 1.2-3.9%, 1.3-3.7%, 1.5-3.5%, 2.6-3.5%, 2.7-3.4%, 2.8-3.3%, 2.9-3.2%, 2.5-3.7% by weight, but it is not limited thereto, and the foregoing caffeine may occupy about 0.5-23% by weight, such as about 0.6-22%, 0.8-20%, 1-18%, 2-16%, 3-15%, 5-12%, 7-10% by weight , but it is not limited thereto.

In one embodiment, for the composition for improving the solubility of poorly soluble substances of the present disclosure, the poorly soluble substance may be a Type III tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, in the composition for improving the solubility of poorly soluble substances of the present disclosure, the foregoing hydroxypropyl-β-cyclodextrin or hydroxypropyl-γ-cyclodextrin may occupy about 68-96% by weight, such as about 69-95.5%, 70-95%, 72-92%, 75-90%, 73-88%, 75-85%, 77-83%, 78-80% by weight, but it is not limited thereto, the foregoing hydroxypropyl methyl cellulose may occupy about 1-4% by weight, such as about 1.1-4%, 1.2-3.9%, 1.3-3.7%, 1.5-3.5%, 2.6-3.3%, 2.7-3.2%, 2.8-3.0%, 2.5-3.4% by weight, but it is not limited thereto, and the foregoing caffeine may occupy about 0.8-30% by weight, such as about 0.9-29.5%, 1-29%, 2-27%, 3-25%, 5-23%, 7-20%, 8-18%, 10-17%, 12-15% by weight , but it is not limited thereto.

Furthermore, in one embodiment, the composition for improving the solubility of poorly soluble substances of the present disclosure mentioned above, in addition to the cyclodextrin and/or a derivative thereof, the at least one water-soluble polymer and the at least one water-soluble stabilizer mentioned above, may further comprise a solvent to form a solution with the cyclodextrin and/or a derivative thereof, the at least one water-soluble polymer and the at least one water-soluble stabilizer mentioned above. In this solution, the total concentration of the cyclodextrin and/or a derivative thereof, the at least one water-soluble polymer and the at least one water-soluble stabilizer may be about 5-55% (w/v), for example, may be 5-10%, 10-20%, 20-25%, 30-35%, 35-40%, 40-45%, 45-50, 50-55, but it is not limited thereto.

The present disclosure also provides a use of a composition for improving the solubility of poorly soluble substances. In the use of a composition for improving the solubility of poorly soluble substances, the said composition may be any composition for improving the solubility of poorly soluble substances of the present disclosure mentioned above. Furthermore, since the description of the poorly soluble substances, the cyclodextrin and/or a derivative thereof, the at least one water-soluble polymer and the at least one water-soluble stabilizer has been described in the relevant paragraphs of the composition for improving the solubility of poorly soluble substances of the present disclosure mentioned above, it will not be described again herein.

In the use of a composition for improving the solubility of poorly soluble substances, compared with dissolving the poorly soluble substances in an aqueous solvent, dissolving the poorly soluble substance in an aqueous solvent together with the said composition improves the solubility of the poorly soluble substances.

The present disclosure may further provide a complex formulation. The complex formulation of the present disclosure mentioned above may comprise, but is not limited to, at least one active ingredient, cyclodextrin and/or a derivative thereof, at least one water-soluble polymer and the at least one water-soluble stabilizer, in which the at least one active ingredient is a hydrophobic compound. In the complex formulation of the present disclosure mentioned above, the content of each component is not particularly limited, and it may be adjusted according to the content of other components, and/or may be adjusted as needed.

In the complex formulation of the present disclosure mentioned above, the cyclodextrin and/or a derivative thereof, the at least one water-soluble polymer and the at least one water-soluble stabilizer can be regarded as the components of the composition for improving the solubility of a poorly soluble substance of the present disclosure mentioned above, which has an effect of improving the solubility of the active ingredient in the complex formulation of the present disclosure.

As needs, the content of each component in the complex formulation of the present disclosure mentioned above may be adjusted according to the content of other components in the complex formulation of the present disclosure mentioned above while the total content of all component will not exceed 100 wt %.

In the complex formulation of the present disclosure mentioned above, the at least one active ingredient mentioned above may occupy about 0.05-10% by weight, for example, may be about 0.05-0.1%, 0.05-0.08%, 0.06-0.1%, 0.1-0.12%, 0.15-0.25%, 0.5-1%, 1-2%, 1-3%, 2-5%, 2-3%, 3-5%, 5-7%, 8-10% by weight, but it is not limited thereto.

In one embodiment, the complex formulation of the present disclosure mentioned above may be a pharmaceutical formulation. In the complex formulation of the present disclosure mentioned above, the active ingredient means a hydrophobic ingredient that has a therapeutic, alleviating and/or prophylactic effect on a disease and/or a symptom, but it is not limited thereto.

As used herein, “a hydrophobic compound” means any substance having solubility in water of less than about 0.01 g/mL, but it is not limited thereto. In one embodiment, the above-mentioned hydrophobic compound may comprise a steroid drug, an aromatic compound with a molecular weight of 100-1000 Da or a combination thereof, etc., but it is not limited thereto. In another embodiment, the above-mentioned hydrophobic compound may comprise a tyrosine kinase inhibitor, but it is not limited thereto.

Examples of the steroid drug may comprise, but it is not limited to, loteprednol etabonate, dexamethasone, dexamethasone phosphate, prednisolone, prednisolone acetate, fluorometholone, 17β-estradiol, 17α-ethinylestradiol, ethinylestradiol 3-methyl ether, estriol, norethindrone, norethindrone acetate, norgestrel, ethisterone, methoxyprogesterone, progesterone, 17-methyltestosterone, triamcinolone, testosterone, spironolactone, alfaxalone, lanostanoid or a combination thereof.

Moreover, the aromatic compound with a molecular weight of 100-1000 Da may comprise axitinib, methotrexate, folic acid, diclofenac sodium, lutein, any combination thereof, etc., but it is not limited thereto.

Furthermore, the tyrosine kinase mentioned above may comprise, but is not limited to, a Type I tyrosine kinase inhibitor, a Type II tyrosine kinase inhibitor, a Type III tyrosine kinase inhibitor, a Type IV tyrosine kinase inhibitor, a Type V tyrosine kinase inhibitor, etc., or a combination thereof. The Type I tyrosine kinase inhibitor mentioned above may comprise cabozantinib, pazopanib, etc., or a combination thereof, but it is not limited thereto. Type II tyrosine kinase inhibitor mentioned above may comprise, axitinib, regorafenib etc., or a combination thereof, but is not limited thereto. The Type III tyrosine kinase inhibitor mentioned above may comprise, binimetinib, etc., but is not limited thereto.

In the complex formulation of the present disclosure mentioned above, the cyclodextrin and/or a derivative thereof may occupy about 40-99.5% by weight, such as 40-45%, 45-50%, 50-55%, 55-60%, 60-65%, 60-65%, 65-70%, 70-75%, 75-80%, 80-85%, 85-90%, 95-99.5%, 55-99.5%, 50-75%, 60-85%, 60-99.5%, 80-99.5% by weight, but it is not limited thereto.

Furthermore, since the description of the cyclodextrin and/or a derivative thereof in the complex formulation has been described in the relevant paragraphs of the composition for improving the solubility of poorly soluble substances of the present disclosure mentioned above, it will not be described again herein.

In the complex formulation of the present disclosure mentioned above, the at least one water-soluble polymer may occupy about 0.05-10% by weight, such as about 0.05-0.1%, 0.05-0.08%, 0.06-0.1%, 0.1-0.12%, 0.15-0.25%, 0.5-1%, 1-2%, 1-3%, 2-4%, 2-5%, 2-3%, 2.5-4%, 3-3.5%, 3-5%, 5-7%, 8-10% by weight, but it is not limited thereto.

Moreover, since the description of the at least one water-soluble polymer in the complex formulation also has been described in the relevant paragraphs of the composition for improving the solubility of poorly soluble substances of the present disclosure mentioned above, it will not be described again herein.

Furthermore, in the complex formulation of the present disclosure mentioned above, the at least one water-soluble stabilizer may occupy about 0.05-60% by weight, such as about 0.05-0.1%, 0.05-0.08%, 0.06-0.1%, 0.1-0.12%, 0.15-0.25%, 0.5-1%, 1-2%, 1-3%, 2-5%, 2-3%, 3-5%, 5-7%, 8-10%, 10-15%, 15-20%, 20-25%, 25-30%, 30-35%, 35-40%, 40-55%, 45-50%, 50-55%, 55-60%, 0.4-30%, 0.4-25%, 0.4-20%, 0.5-30%, 0.5-25%, 0.5-20%, 1-30%, 1-25%, 1-20%, 1-15%, 1-10%, 2-30%, 2-25%, 2-20%, 3-30%, 3-25%, 3-20%, 4-30%, 4-25%, 4-20%, 4-10%, 4.5-25%, 5-20%, 6-15%, 7-25%, 8-30%, 9-30%, 10-20%, 15-25%, 20-30%, 0.4-4%, 0.5-3.5%, 0.6-3%, 0.7-2.5%, 0.8-2.0%, 0.9-1.5%, 1-1.2%, 1.2-2%, 1.5-2.5% by weight, but it is not limited thereto

Similarly, since the description of the at least one water-soluble stabilizer in the complex formulation has been described in the relevant paragraphs of the composition for improving the solubility of poorly soluble substances of the present disclosure mentioned above, it will not be described again herein.

In one embodiment, in the complex formulation of the present disclosure mentioned above, the at least one water-soluble stabilizer mentioned above is the amino acid with a polar side chain or the oligopeptide containing at least one amino acid with a polar side chain, and the active ingredient mentioned above may occupy about 0.5-5% by weight, cyclodextrin and/or a derivative thereof mentioned above may occupy about 40-85% by weight, the at least one water-soluble polymer mentioned above may occupy about 0.5-5%, by weight, and the amino acid with a polar side chain or the oligopeptide containing at least one amino acid with a polar side chain mentioned may occupy about 15-55% by weight. Furthermore, the active ingredient mentioned above may be loteprednol etabonate or axitinib, the cyclodextrin and/or a derivative thereof mentioned above may be hydroxypropyl-γ-cyclodextrin, and the at least one water-soluble polymer mentioned above may be hydroxypropyl methyl cellulose.

Furthermore, in the complex formulation of the present disclosure mentioned above, for one specific embodiment, under the premise that the at least one water-soluble stabilizer mentioned above is the amino acid with a polar side chain or the oligopeptide containing at least one amino acid with a polar side chain, and the cyclodextrin and/or a derivative thereof, the at least one water-soluble polymer, and the amino acid with a polar side chain or the oligopeptide containing at least one amino acid with a polar side chain may respectively occupy about 40-85% by weight, 0.5-5% by weight, and 15-55% by weight, if it is further limited to that the active ingredient may be loteprednol etabonate or axitinib, the cyclodextrin and/or a derivative thereof may be hydroxypropyl-γ-cyclodextrin, the at least one water-soluble polymer may be hydroxypropyl methyl cellulose, and the at least one water-soluble stabilizer may be amino acid with a polar side chain, and the amino acid with a polar side chain may comprise glutamine, glutamic acid or histidine, in this specific embodiment, the foregoing loteprednol etabonate or axitinib may occupy about 0.1-3%, the foregoing hydroxypropyl-γ-cyclodextrin may occupy about 70-85%, the foregoing hydroxypropyl methyl cellulose may occupy about 0.5-3%, and the foregoing amino acid with a polar side chain may occupy about 10-25%.

Alternatively, in the complex formulation of the present disclosure mentioned above, for one specific embodiment, under the premise that the at least one water-soluble stabilizer mentioned above is the amino acid with a polar side chain or the oligopeptide containing at least one amino acid with a polar side chain, the cyclodextrin and/or a derivative thereof, the at least one water-soluble polymer, and the amino acid with a polar side chain or the oligopeptide containing at least one amino acid with a polar side chain may respectively occupy about 40-85%, 0.5-5%, and 15-55% by weight, if it is further limited to that the active ingredient may be loteprednol etabonate or axitinib, the cyclodextrin and/or a derivative thereof may be hydroxypropyl-γ-cyclodextrin, the at least one water-soluble polymer may be hydroxypropyl methyl cellulose, and the at least one water-soluble stabilizer may be oligopeptide containing at least one amino acid with a polar side chain, and oligopeptide containing at least one amino acid with a polar side chain may comprise carnosine, glutathione or leucine-glycine-glycine, in this specific embodiment, the foregoing loteprednol etabonate or axitinib may occupy about 0.1-3% by weight, the foregoing hydroxypropyl-γ-cyclodextrin may occupy about 40-80% by weight, the foregoing hydroxypropyl methyl cellulose may occupy about 0.5-3% by weight, and the foregoing oligopeptide containing at least one amino acid with a polar side chain may occupy about 15-55% by weight.

In another embodiment, in the complex formulation of the present disclosure mentioned above, the at least one water-soluble stabilizer mentioned above may be the derivative of purine, and the derivative of purine may be caffeine, and the active ingredient mentioned above may be loteprednol etabonate or axitinib, the cyclodextrin and/or a derivative thereof mentioned above may be hydroxypropyl-γ-cyclodextrin, and the at least one water-soluble polymer mentioned above may be hydroxypropyl methyl cellulose. In one specific embodiment of this embodiment, the foregoing loteprednol etabonate or axitinib may occupy about 1.5-5% by weight, the foregoing hydroxypropyl-γ-cyclodextrin may occupy about 70-99.5% by weight, the foregoing hydroxypropyl methyl cellulose may occupy about 0.1-5% by weight, and the foregoing caffeine may occupy about 0.05-20% by weight.

Furthermore, the complex formulation of the present disclosure, in addition to the active ingredient, the cyclodextrin and/or a derivative thereof, the at least one water-soluble polymer and the at least one water-soluble stabilizer, may further comprise a surfactant to make the complex formulation form a microparticle. In the complex formulation of the present disclosure mentioned above, the at least one active ingredient may occupy about 0.05-10% by weight, the cyclodextrin and/or a derivative thereof may occupy about 40-99.5% by weight, the at least one water-soluble polymer may occupy by weight, the water-soluble stabilizer may occupy 0.05-60% by weight, and the 0.05-10%, surfactant may occupy 0.05-10% by weight, but it is not limited thereto.

The surfactant may comprise, but is not limited to Tween 80, Tween 20, Span 80, DSPE-PEG, a derivative of DSPE-PEG or a combination thereof. In one embodiment, the surfactant may be Tween 80. In another embodiment, the surfactant may be a combination of Tween 80 and DSPE-PEG.

The average particle size of the microparticle may be 500 nm-100 μm, for example, may be about 500 nm-800 nm, 800 nm-1000 nm, 10 μm-50 μm, 50 μm-100 μm, but it is not limited thereto.

In one embodiment, the at least one active ingredient may be loteprednol etabonate or axitinib, and the surfactant may be Tween 80. Moreover, in this embodiment, the loteprednol etabonate or axitinib may occupy about 0.01-10% by weight, the cyclodextrin and/or a derivative thereof may occupy about 50-90% by weight, the at least one water-soluble polymer may occupy 0.05-20% by weight, the water-soluble stabilizer may occupy 0.05-20% by weight, and Tween 80 may occupy 0.1-10% by weight, but it is not limited thereto.

Moreover, the complex formulation of the present disclosure, in addition to the active ingredient, the cyclodextrin and/or a derivative thereof, the at least one water-soluble polymer and the at least one water-soluble stabilizer mentioned above, may further comprise a solvent to form a liquid dosage form with the at least one active ingredient, the cyclodextrin and/or a derivative thereof, the at least one water-soluble polymer and the at least one water-soluble stabilizer. In this liquid dosage form, the total concentration of the cyclodextrin and/or a derivative thereof, the at least one water-soluble polymer and the at least one water-soluble stabilizer may be about 5-55% (w/v), for example, may be 5-10%, 10-20%, 20-25%, 30-35%, 35-40%, 40-45%, 45-50%, 50-55%, but it is not limited thereto.

The solvent mentioned above may comprise, but is not limited to water, ethanol or a water/ethanol mixture, etc.

In one embodiment, the complex formulation may form a liquid dosage form, and the complex formulation may be a pharmaceutical formulation. The type of the liquid dosage form mentioned above may comprise, but is not limited to, an oral dosage form, an injection dosage form, an eye drop, etc. Moreover, examples of the injection dosage form mentioned above may comprise, but is not limited to, a subcutaneous injection dosage form, an intramuscular injection dosage form or an intraperitoneal injection dosage form. In one embodiment, the liquid dosage form of the complex formulation of the present disclosure is an eye drop.

In addition, the complex formulation may be administered to a subject in need of the complex formulation, but it is not limited thereto. The administration route of the complex formulation of the present disclosure may be administered parenterally, orally, by an inhalation spray, or via an implanted reservoir, but is not limited thereto. The parenteral methods may comprise, but is not limited to, smearing the affected regions, subcutaneous, intracutaneous, intravenous, intramuscular, intra-articular, intra-arterial, intrasynovial, intrasternal, intrathecal, and intralesional injection, intraocular injection, eye drops, as well as infusion techniques, etc.

Furthermore, the foregoing subject may include, but is not limited to, a vertebrate. The vertebrate mentioned above may include a fish, an amphibian, a reptile, a bird, or a mammal, but it is not limited thereto. Examples of mammals include, but are not limited to, a human, an orangutan, a monkey, a horse, a donkey, a dog, a cat, a rabbit, a guinea pig, a rat, and a mouse. In one embodiment, the subject is a human.

Moreover, the present disclosure may provide another complex formulation. The complex formulation mentioned above may comprise, but is not limited to, at least one active ingredient and any foregoing composition for improving the solubility of poorly soluble substances of the present disclosure. In the complex formulation mentioned above, the content of each component is not particularly limited, and it may be adjusted according to the content of other components, and/or may be adjusted as needed.

For example, for the complex formulation mentioned above, the content of at least one active ingredient may be about 0.05-20 wt %, such as about 0.06-20 wt %, 0.1-20 wt %, 0.2-15 wt %, 0.5-12 wt %, 0.6-10 wt %, 0.8-8, 1-5 wt %, 2.5-4.5 wt %, 3-4 wt %, but it is not limited thereto.

In one embodiment, the at least one active ingredient in the foregoing complex formulation may be a hydrophobic compound. In another embodiment, the at least one active ingredient in the foregoing complex formulation may mean a hydrophobic ingredient that has a therapeutic, alleviating and/or prophylactic effect on a disease and/or a symptom, but it is not limited thereto. In one specific embodiment, the at least one active ingredient may comprise a tyrosine kinase inhibitor which is a hydrophobic ingredient, but it is not limited thereto. The tyrosine kinase mentioned above may comprise, but is not limited to, a Type I tyrosine kinase inhibitor, a Type II tyrosine kinase inhibitor, a Type III tyrosine kinase inhibitor, a Type IV tyrosine kinase inhibitor, a Type V tyrosine kinase inhibitor, etc., or a combination thereof In addition, the Type I tyrosine kinase inhibitor mentioned above may comprise cabozantinib, pazopanib, etc., or a combination thereof, but it is not limited thereto. Type II tyrosine kinase inhibitor mentioned above may comprise, axitinib, regorafenib etc., or a combination thereof, but is not limited thereto. The Type III tyrosine kinase inhibitor mentioned above may comprise, binimetinib, etc., but is not limited thereto.

Moreover, in one embodiment, for the composition for improving the solubility of poorly soluble substances in the complex formulation mentioned above, the at least one water-soluble stabilizer mentioned above may be caffeine. In this embodiment, for the complex formulation of the present disclosure, the contents of the foregoing cyclodextrin and/or a derivative thereof, the foregoing at least one water-soluble polymer and caffeine in the foregoing composition for improving the solubility of poorly soluble substances are described in the following. The foregoing cyclodextrin and/or a derivative thereof may occupy about 60-97% by weight, such as about 60-96.5%, 65-95%, 70-90%, 75-85% by weight, but it is not limited thereto. The foregoing at least one water-soluble polymer may occupy about 0.5-4% by weight, such as about 0.55-4%, 0.6-3.95%, 0.7-3.9%, 0.8-3.85%, 0.9-3.8%, 1-4%, 1.1-3.9%, 1.2-3.8%, 1.5-3.6%, 1.8-3.5%, 2.0-3.4%, 2.2-3.2%, 2.4-3.0%, 2.6-2.8%, 2-4%, 2.5-3.5% by weight, but it is not limited thereto. The caffeine may occupy about 0.4-30% by weight, such as about 0.4-25%, 0.4-20%, 0.5-30%, 0.5-25%, 0.5-20%, 1-30%, 1-25%, 1-20%, 1-15%, 1-10%, 2-30%, 2-25%, 2-20%, 3-30%, 3-25%, 3-20%, 4-30%, 4-25%, 4-20%, 4-10%, 4.5-25%, 5-20%, 6-15%, 7-25%, 8-30%, 9-30%, 10-20%, 15-25%, 20-30%, 0.4-4%, 0.5-3.5%, 0.6-3%, 0.7-2.5%, 0.8-2.0%, 0.9-1.5%, 1-1.2%, 1.2-2%, 1.5-2.5% by weight, but it is not limited thereto.

Furthermore, in one embodiment, for the composition for improving the solubility of poorly soluble substances in the complex formulation mentioned above, the cyclodextrin and/or a derivative thereof mentioned above may be hydroxypropyl-β-cyclodextrin or hydroxypropyl-γ-cyclodextrin, the at least one water-soluble polymer mentioned above may be hydroxypropyl methyl cellulose and the at least one water-soluble stabilizer mentioned above may be caffeine. For the complex formulation of the present disclosure mentioned above, the foregoing hydroxypropyl-β-cyclodextrin or hydroxypropyl-γ-cyclodextrin may occupy about 60-97% by weight in the foregoing composition for improving the solubility of poorly soluble substances, such as about 60-96.5%, 65-95%, 70-90%, 75-85% by weight, but it is not limited thereto. For the complex formulation of the present disclosure mentioned above, the foregoing hydroxypropyl methyl cellulose may occupy about 0.5-4% by weight in the foregoing composition for improving the solubility of poorly soluble substances, such as about 0.55-4%, 0.6-3.95%, 0.7-3.9%, 0.8-3.85%, 0.9-3.8%, 1-4%, 1.1-3.9%, 1.2-3.8%, 1.5-3.6%, 1.8-3.5%, 2.0-3.4%, 2.2-3.2%, 2.4-3.0%, 2.6-2.8%, 2-4%, 2.5-3.5% by weight, but it is not limited thereto. For the complex formulation of the present disclosure mentioned above, the caffeine may occupy about 0.4-30% by weight in the foregoing composition for improving the solubility of poorly soluble substances, such as about 0.4-25%, 0.4-20%, 0.5-30%, 0.5-25%, 0.5-20%, 1-30%, 1-25%, 1-20%, 1-15%, 1-10%, 2-30%, 2-25%, 2-20%, 3-30%, 3-25%, 3-20%, 4-30%, 4-25%, 4-20%, 4-10%, 4.5-25%, 5-20%, 6-15%, 7-25%, 8-30%, 9-30%, 10-20%, 15-25%, 20-30%, 0.4-4%, 0.5-3.5%, 0.6-3%, 0.7-2.5%, 0.8-2.0%, 0.9-1.5%, 1-1.2%, 1.2-2%, 1.5-2.5% by weight, but it is not limited thereto.

In one embodiment, for the complex formulation of the present disclosure, the poorly soluble substance may be a Type I tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, for the complex formulation of the present disclosure mentioned above, the foregoing hydroxypropyl-β-cyclodextrin or hydroxypropyl-γ-cyclodextrin may occupy about 65-97% by weight in the composition for improving the solubility of poorly soluble substances mentioned above, such as about 66-96.9%, 70-96.5%, 75-95%, 80-92% by weight, but it is not limited thereto, the foregoing hydroxypropyl methyl cellulose may occupy about 1-4% by weight in the composition for improving the solubility of poorly soluble substances mentioned above, such as about 1.1-4%, 1.2-3.9%, 1.3-3.7%, 1.5-3.5%, 2.1-3.6%, 2.5-3.5%, 2.7-3.5%, 2.8-3.0%, 2-4% by weight, but it is not limited thereto, and the foregoing caffeine may occupy about 0.7-29.5% by weight in the composition for improving the solubility of poorly soluble substances mentioned above, such as about 0.75-29%, 0.8-28.5%, 3-25%, 5-22%, 8-20%, 10-18%, 12-16% by weight , but it is not limited thereto.

In one embodiment, for the complex formulation of the present disclosure, the poorly soluble substance may be a Type II tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, for the complex formulation of the present disclosure mentioned above, the foregoing hydroxypropyl-β-cyclodextrin or hydroxypropyl-γ-cyclodextrin may occupy about 74-96% by weight in the composition for improving the solubility of poorly soluble substances mentioned above, such as about 75-95%, 77-93%, 80-92%, 82-90%, 83-88% by weight, but it is not limited thereto, the foregoing hydroxypropyl methyl cellulose may occupy about 1-4% by weight in the composition for improving the solubility of poorly soluble substances mentioned above, such as about 1.1-4%, 1.2-3.9%, 1.3-3.7%, 1.5-3.5%, 2.6-3.5%, 2.7-3.4%, 2.8-3.3%, 2.9-3.2%, 2.5-3.7% by weight, but it is not limited thereto, and the foregoing caffeine may occupy about 0.5-23% by weight in the composition for improving the solubility of poorly soluble substances mentioned above, such as about 0.6-22%, 0.8-20%, 1-18%, 2-16%, 3-15%, 5-12%, 7-10% by weight , but it is not limited thereto.

In one embodiment, for the complex formulation of the present disclosure, the poorly soluble substance may be a Type III tyrosine kinase inhibitor, the at least one water-soluble stabilizer mentioned above may be caffeine, the cyclodextrin and/or a derivative thereof mentioned above may be hydroxypropyl-β-cyclodextrin or hydroxypropyl-γ-cyclodextrin, and the at least one water-soluble polymer mentioned above may be hydroxypropyl methyl cellulose. In this embodiment, for the complex formulation of the present disclosure mentioned above, the foregoing hydroxypropyl-β-cyclodextrin or hydroxypropyl-γ-cyclodextrin may occupy about 68-96% by weight in the composition for improving the solubility of poorly soluble substances mentioned above, such as about 69-95.5%, 70-95%, 72-92%, 75-90%, 73-88%, 75-85%, 77-83%, 78-80% by weight, but it is not limited thereto, the foregoing hydroxypropyl methyl cellulose may occupy about 1-4% by weight in the composition for improving the solubility of poorly soluble substances mentioned above, such as about 1.1-4%, 1.2-3.9%, 1.3-3.7%, 1.5-3.5%, 2.6-3.3%, 2.7-3.2%, 2.8-3.0%. 2.5-3.4% by weight, but it is not limited thereto, and the foregoing caffeine may occupy about 0.8-30% by weight in the composition for improving the solubility of poorly soluble substances mentioned above, such as about 0.9-29.5%, 1-29%, 2-27%, 3-25%, 5-23%, 7-20%, 8-18%, 10-17%, 12-15% by weight , but it is not limited thereto.

The present disclosure may provide a liquid formulation of an active ingredient. The liquid formulation of an active ingredient of the present disclosure mentioned above may comprise, but is not limited to, at least one active ingredient, any foregoing composition for improving the solubility of poorly soluble substances of the present disclosure and a solvent. In the liquid formulation of an active ingredient of the present disclosure mentioned above, the content of each component is not particularly limited, and it may be adjusted according to the content of other components, and/or may be adjusted as needed.

For example, the content of the at least one active ingredient in the liquid formulation of an active ingredient mentioned above may be about 0.01-10% (w/v), such as about 0.02-8% (w/v), 0.05-5% (w/v), 0.1-4% (w/v), 0.2-3.5% (w/v), 0.3-3% (w/v), 0.4-2.5% (w/v), 0.5-2% (w/v), 0.6-1% (w/v), 1-10% (w/v), 2-8% (w/v), 3-5% (w/v), 4-6% (w/v), but it is not limited thereto.

The related description for the at least one active ingredient in the foregoing liquid formulation of an active ingredient may refer to that for the at least one active ingredient in the foregoing complex formulation, and thus is not repeated herein.

Similarly, the related description for the composition for improving the solubility of poorly soluble substances of the present disclosure in the foregoing liquid formulation of an active ingredient may refer to that for the composition for improving the solubility of poorly soluble substances of the present disclosure in the foregoing complex formulation, and thus is not repeated herein.

The solvent in the foregoing liquid formulation may comprise, but is not limited to water, ethanol or a water/ethanol mixture, etc.

In one embodiment, the foregoing liquid formulation of an active ingredient may further comprise a surfactant to form microparticles in the liquid formulation of an active ingredient form. The content of the surfactant in the liquid formulation of an active ingredient mentioned above may be about 0.01-2% (w/v), such as about 0.02-1.8% (w/v), 0.03-1.5% (w/v), 0.05-1.2% (w/v), 0.1-1% (w/v), 0.2-0.8% (w/v), 0.3-0.6% (w/v), but it is not limited thereto.

The surfactant may comprise, but is not limited to Tween 80, Tween 20, Span 80, DSPE-PEG, a derivative of DSPE-PEG or a combination thereof. In one embodiment, the surfactant may be Tween 80. In another embodiment, the surfactant may be a combination of Tween 80 and DSPE-PEG.

The average particle size of the microparticle mentioned above may be 500 nm-100 μm, for example, may be about 500 nm-800 nm, 800 nm-1000 nm, 10 μm-50 μm, 50 μm-100 μm, but it is not limited thereto.

In addition, in one embodiment, the liquid formulation of an active ingredient mentioned above may be a liquid dosage form of pharmaceutical formulation.

The liquid dosage form of pharmaceutical formulation mentioned above may comprise, but is not limited to, an oral dosage form, an injection dosage form, an eye drop, etc. Furthermore, examples of the injection dosage form mentioned above may comprise, but is not limited to, a subcutaneous injection dosage form, an intramuscular injection dosage form or an intraperitoneal injection dosage form. In one specific embodiment, the liquid dosage form of pharmaceutical formulation mentioned above is an eye drop.

The liquid formulation of an active ingredient may be administered to a subject in need of the liquid formulation of an active ingredient mentioned above, but it is not limited thereto. The administration route of the liquid formulation of an active ingredient may be administered parenterally, orally, by an inhalation spray, or via an implanted reservoir, but is not limited thereto. The parenteral methods may comprise, but is not limited to, smearing the affected regions, subcutaneous, intracutaneous, intravenous, intramuscular, intra-articular, intra-arterial, intrasynovial, intrasternal, intrathecal, and intraleaional injection, intraocular injection, eye drops, as well as infusion techniques, etc.

Furthermore, the foregoing subject may include, but is not limited to, a vertebrate. The vertebrate mentioned above may include a fish, an amphibian, a reptile, a bird, or a mammal, but it is not limited thereto. Examples of mammals include, but are not limited to, a human, an orangutan, a monkey, a horse, a donkey, a dog, a cat, a rabbit, a guinea pig, a rat, and a mouse. In one embodiment, the subject is a human.

### EXAMPLES

### Example 1

### Solubility Test for Loteprednol Etabonate (LE)

### Example 1-1

Effect of using different amino acids, oligopeptides or monosaccharide as a stabilizer of a formulation on the solubility of the drug (loteprednol etabonate) in a formulation containing loteprednol etabonate (LE)/hydroxypropyl-γ-cyclodextrin (HPγCD)

### 1. Method

The samples were formulated and analyzed according to the formulas shown in the following Table 1 and the methods described below at room temperature.

Hydroxypropyl-γ-cyclodextrin, a water-soluble polymer (hydroxypropyl methyl cellulose (HPMC (molecular weight: 16676)) and an amino acid (glutamine (Gln), glutamic acid (Glu) or histidine (His)) or an oligopeptide (glutathione (GSH), L-carnosine or leucine-glycine-glycine (Leu-Gly-Gly)) as a stabilizer of the formulation were dissolved in 3 mL of deionized water to form a solution, and groups using an oligopeptide (glycine-glycine (Gly-Gly)) and monosaccharide (mannitol) as stabilizers of the formulations were served as the negative control groups.

Next, in an ultrasonic water bath environment, the above solution was slowly added to a methanol solution containing 4 mg loteprednol etabonate (LE) (10 mg LE/mL) to form a mixture. After that, the mixture was dried in a vacuum environment to performing drying to remove the solvent therein and obtain a dried product.

Thereafter, the dried product was re-dissolved in 2 mL of deionized water to form a test sample, and the test sample solution was adjusted to pH 5.5 with 1 M NaOH. Next, the test sample was filtered with a 0.22 μm pore size filter to remove undissolved precipitate. Finally, the content of loteprednol etabonate (LE) in the test sample was analyzed by high performance liquid chromatography (HPLC).

TABLE 1

| | LE | HPγCD | HPMC | Stabilizer |
|---|---|---|---|---|
| Sample | (mg) | (mg) | (mg) | (mg) |
| LE | 4 | 0 | 0 | 0 |
| LE/GSH | 4 | 0 | 0 | 120 |
| LE/HPMC | 4 | 0 | 5 | 0 |
| LE/HPγCD | 4 | 204 | 0 | 0 |
| LE/HPγCD/HPMC | 4 | 204 | 5 | 0 |
| LE/HPγCD/GSH | 4 | 204 | 0 | 120 |
| LE/HPMC/GSH | 4 | 0 | 5 | 120 |
| LE/HPγCD/HPMC/GSH | 4 | 204 | 5 | 120 |
| LE/HPγCD/HPMC/Gln | 4 | 204 | 5 | 57.06 |
| LE/HPγCD/HPMC/Glu | 4 | 204 | 5 | 57.44 |
| LE/HPγCD/HPMC/His | 4 | 204 | 5 | 60.59 |
| LE/HPγCD/HPMC/Carnosine | 4 | 204 | 5 | 88.34 |
| LE/HPγCD/HPMC/Leu-Gly-Gly | 4 | 204 | 5 | 95.78 |
| LE/HPγCD/HPMC/Gly-Gly | 4 | 204 | 5 | 51.58 |
| LE/HPγCD/HPMC/Mannitol | 4 | 204 | 5 | 71.13 |

### 2. Results

The solubility of loteprednol etabonate (LE) of each sample and the degree of solubility improvement compared to loteprednol etabonate (LE)/HPγCD are as shown in Table 2 below.

TABLE 2

| | | | Fold of |
|---|---|---|---|
| | | Solubility | solubility |
| | LE feeding | of LE | relative to |
| Sample | (μg) | (μg/mL) | LE/HPγCD |
| LE | 4000 | 1.5 | N.A. |
| LE/GSH | 4000 | 1.5 | N.A. |
| LE/HPMC | 4000 | 3.4 | N.A. |
| LE/HPγCD | 4000 | 290.7 | 1.00 |
| LE/HPγCD/HPMC | 4000 | 1010.6 | 3.48 |
| LE/HPγCD/GSH | 4000 | 331.4 | 1.14 |
| LE/HPMC/GSH | 4000 | 1.8 | N.A. |
| LE/HPγCD/HPMC/GSH | 4000 | 1885.4 | 6.49 |
| LE/HPγCD/HPMC/Gln | 4000 | 1806.1 | 6.21 |
| LE/HPγCD/HPMC/Glu | 4000 | 1705.5 | 5.87 |
| LE/HPγCD/HPMC/His | 4000 | 1634.2 | 5.62 |
| LE/HPγCD/HPMC/Carnosine | 4000 | 1827.9 | 6.29 |
| LE/HPγCD/HPMC/Leu-Gly-Gly | 4000 | 1551.2 | 5.34 |
| LE/HPγCD/HPMC/Gly-Gly | 4000 | 800.0 | 2.75 |
| LE/HPγCD/HPMC/Mannitol | 4000 | 1025.0 | 6.60 |

N.A.: Not detected

According to Table 2 above, it can be known that the water solubility of LE is extremely low, only 1.5 μg/mL. Similarly, when LE is only mixed with the water-soluble polymer, HPMC, or the formulation stabilizer, GSH, the solubility of LE in an aqueous solution still cannot be effectively improved. Only mixing LE with HPγCD can improve the solubility of LE in an aqueous solution, and that increases from less than 4 μg/mL to 290 μg/mL.

Moreover, when the formula of LE/HPγCD was further mixed with the water-soluble polymer, HPMC, or the formulation stabilizer, GSH, respectively, the LE solubility may be about 1.2-3.5 times that of the combination of LE/HPγCD.

Furthermore, when LE was mixed together with HPγCD, the water-soluble polymer, HPMC, and the formulation stabilizer (amino acid or oligopeptide), the solubility of LE could be further improved, for example, LE solubility of LE/HPγCD/HPMC/GSH formula might be 6.5 times that of the LE/HPγCD formula.

In addition, according to the test results of a plurality of different formulations, it is shown that specific amino acids and oligopeptides can effectively improve the solubility of LE in the formulation, and among them, using glutamine and glutamic acid (amino acid) and carnosine and glutathione (oligopeptide) as a formulation stabilizer can significantly improve the solubility of LE which may be about 5.3-6.6 times that of LE/HPγCD. In contrast, the stability of the sample, LE/HPγCD/HPMC/mannitol, was not good, and it precipitated rapidly within 1 hour.

### Example 1-2

Effect of using histidine (His) as a stabilizer of a formulation on the solubility of the drug (loteprednol etabonate) in a formulation containing loteprednol etabonate (LE)/hydroxypropyl-γ-cyclodextrin (HPγCD)

### 1. Method

The samples were formulated and analyzed according to the formulas shown in the following Table 3 and the methods described below at room temperature.

Hydroxypropyl-γ-cyclodextrin, a water-soluble polymer (hydroxypropyl methyl cellulose (HPMC) (molecular weight: 16676)), and histidine (His) as a stabilizer of the formulation were co-dissolved in secondary water to form a solution. Next, the above solution was slowly added to a methanol solution containing loteprednol etabonate (LE) to form a mixture. After that, the mixture was treated by a rotary evaporator to completely remove methanol, and the pH was adjusted to 5.5 with a 0.1 M HCl aqueous solution, and the final solution volume was fixed to 1 mL (the insufficient portion was replenished with secondary water) to form a test sample. Thereafter, the test sample was then filtered with a 0.22 μm pore size filter to remove undissolved precipitate. Finally, the content of loteprednol etabonate (LE) in the test sample was analyzed by high performance liquid chromatography (HPLC).

TABLE 3

| | | | | | | Solubility |
|---|---|---|---|---|---|---|
| | | LE | HPγCD | His | HPMC | of LE |
| Number | Sample | (mg) | (mg) | (mg) | (mg) | (μg/mL) |
| 1 | LE | 8 | — | — | — | 1.5 |
| 2 | LE/2HPγCD/His/ | 8 | 408 | — | — | 807.1 |
| | HPMC | | | | | |
| 3 | LE/2HPγCD/His/ | 6 | 408 | 80 | 10 | 5162.7 |
| | HPMC | | | | | |

—: No addition

### 2. Results

The solubility of loteprednol etabonate (LE) of each sample and the degree of solubility improvement compared to loteprednol etabonate (LE)/HPγCD are as shown in Table 4 below.

TABLE 4

| | | The amount | |
|---|---|---|---|
| | | of LE that | |
| | | can be loaded | Fold of |
| | | per unit of | solubility |
| | | HPγCD | relative to |
| Number | Sample | (μg/mg) | LE/2HPγCD |
| 1 | LE | N.A. | N.A. |
| 2 | LE/2HPγCD | 2.0 | 1.0 |
| 3 | LE/2HPγCD/ | 12.7 | 6.4 |
| | His/HPMC | | |

N.A.: Not detected

According to Table 4, it can be known that in the LE/2HPγCD (Sample 2) formulation, the range of the amount of LE that can be loaded per unit of HPγCD is about 2.0 (μg/mg). However, when the water-soluble polymer, HPMC, and the formulation stabilizer, histidine, (LE/2HPγCD/His/HPMC, Sample 3) were added, the range of the amount of drug that can be loaded per unit of HPγCD was significantly increased to 12.7 (μg/mg). Namely, the solubility of LE added to the formulation of the water-soluble polymer, HPMC, and the formulation stabilizer, histidine, was 6.4 times that of Sample 2 (LE/2HPγCD formulation).

### Example 1-3

Effect of using glutathione (GSH) as a stabilizer of a formulation on the solubility of the drug (loteprednol etabonate) in a formulation containing loteprednol etabonate (LE)/hydroxypropyl-γ-cyclodextrin (HPγCD)

### 1. Method

The samples were formulated and analyzed according to the formula shown in the following Table 5 and the methods described below at room temperature.

Hydroxypropyl-γ-cyclodextrin, a water-soluble polymer (hydroxypropyl methyl cellulose (HPMC) (molecular weight: 16676)), and glutathione (GSH) as a stabilizer of the formulation were co-dissolved in secondary water to form a solution.

Next, the above solution was slowly added to a methanol solution containing loteprednol etabonate (LE) to form a mixture. After that, the mixture was treated by a rotary evaporator to completely remove methanol, and the pH was adjusted to 5.5 with a 1 M NaOH aqueous solution, and the final solution volume was fixed to 1 mL (the insufficient portion was replenished with secondary water) to form a test sample. Thereafter, the test sample was then filtered with a 0.22 μm pore size filter to remove undissolved precipitate. Finally, the content of loteprednol etabonate (LE) in the test sample was analyzed by high performance liquid chromatography (HPLC).

TABLE 5

| | | | | | | Solubility |
|---|---|---|---|---|---|---|
| | | LE | HPγCD | GSH | HPMC | of LE |
| Number | Sample | (mg) | (mg) | (mg) | (mg) | (μg/mL) |
| 1 | LE | 4 | — | — | — | 1.5 |
| 2 | LE/GSH | 4 | — | 240 | — | 2.0 |
| 3 | LE/HPMC | 4 | — | — | 5 | 3.4 |
| 4 | LE/HPγCD | 4 | 204 | — | — | 290.7 |
| 5 | LE/HPγCD/ | 4 | 204 | 160 | 5 | 1517.1 |
| | GSH/HPMC | | | | | |
| 6 | LE/HPγCD/ | 4 | 204 | 240 | 5 | 1080.4 |
| | 1.5GSH/HPMC | | | | | |

—: No addition

### 2. Results

The solubility of loteprednol etabonate (LE) of each sample and the degree of solubility improvement compared to loteprednol etabonate (LE)/HPγCD are as shown in Table 6 below.

TABLE 6

| | | The amount | |
|---|---|---|---|
| | | of LE that | |
| | | can be loaded | Fold of |
| | | per unit of | solubility |
| | | HPγCD | relative to |
| Number | Sample | (μg/mg) | LE/HPγCD |
| 1 | LE | N.A. | N.A. |
| 2 | LE/GSH | N.A. | N.A. |
| 3 | LE/HPMC | N.A. | N.A. |
| 4 | LE/HPγCD | 1.4 | 1.0 |
| 5 | LE/HPγCD/GSH/HPMC | 7.4 | 5.2 |
| 6 | LE/HPγCD/1.5GSH/HPMC | 5.3 | 3.7 |

N.A.: Not detected

According to Table 6, it can be known that in the LE/HPγCD (Sample 4) formulation, the range of the amount of LE that can be loaded per unit of HPγCD is about 1.4 (μg/mg). However, when the water-soluble polymer, HPMC, and the formulation stabilizer, glutathione (GSH), (Sample 5 and Sample 6) were added, the range of the amount of drug that can be loaded per unit of HPγCD was significantly increased to 5.3-7.4 (μg/mg). Namely, the solubility of LE added to the formulation of the water-soluble polymer, HPMC, and the formulation stabilizer, glutathione (GSH), was about 3.7-5.2 times that of Sample 4 (LE/HPγCD formulation).

### Example 1-4

Effect of using caffeine as a stabilizer of a formulation on the solubility of the drug (loteprednol etabonate) in a formulation containing loteprednol etabonate (LE)/hydroxypropyl-γ-cyclodextrin (HPγCD)

### 1. Method

The samples were formulated and analyzed according to the formula shown in the following Table 7 and the methods described below at room temperature.

Hydroxypropyl-γ-cyclodextrin, a water-soluble polymer (hydroxypropyl methyl cellulose (HPMC), and caffeine as a stabilizer of the formulation were co-dissolved in secondary water to form a solution.

Next, the above solution was slowly added to a methanol solution containing loteprednol etabonate (LE) to form a mixture. After that, the mixture was treated by a rotary evaporator to completely remove methanol, and the pH was adjusted to 5.5 with a 0.1 M citric acid aqueous solution, and the final solution volume was fixed to 1 mL (the insufficient portion was replenished with secondary water) to form a test sample. Thereafter, the test sample was then filtered with a 0.22 μm pore size filter to remove undissolved precipitate. Finally, the content of loteprednol etabonate (LE) in the test sample was analyzed by high performance liquid chromatography (HPLC).

TABLE 7

| | | | | | | | LE |
|---|---|---|---|---|---|---|---|
| Sample | LE | HPγCD | Caffeine | HPMC | | Volume | Content |
| number | (mg) | (mg) | (mg) | (mg) | pH | (mL) | (μg/mL) |
| 1 | 8 | — | — | — | 5.5 | 1 | 1.5 |
| 2 | 8 | 102 | — | — | 5.5 | 1 | 138.0 |
| 3 | 8 | 204 | — | — | 5.5 | 1 | 341.0 |
| 4 | 8 | 408 | — | — | 5.5 | 1 | 807.1 |
| 5 | 12 | 408 | 50 | 10 | 5.5 | 1 | 5173.8 |
| 6 | 12 | 408 | 5 | 10 | 5.5 | 1 | 6706.7 |
| 7 | 12 | 408 | 1 | 10 | 5.5 | 1 | 5622.5 |
| 8 | 12 | 408 | 5 | — | 5.5 | 1 | 684.1 |
| 9 | 12 | 408 | 5 | 2 | 5.5 | 1 | 5184.0 |

—: No addition

### 2. Results

The solubility of loteprednol etabonate (LE) of each sample and the degree of solubility improvement compared to loteprednol etabonate (LE)/HPγCD are as shown in Table 8 below.

TABLE 8

| | The amount | |
|---|---|---|
| | of LE that | |
| | can be loaded | Fold of |
| | per unit of | solubility |
| Sample | HPγCD | relative to |
| number | (μg/mg) | LE/HPγCD |
| 1 | N.A. | N.A. |
| 2 | 1.4 | 0.7 |
| 3 | 1.7 | 0.9 |
| 4 | 2.0 | 1.0 |
| 5 | 12.7 | 6.4 |
| 6 | 16.4 | 8.2 |
| 7 | 13.8 | 6.9 |
| 8 | 1.7 | 0.9 |
| 9 | 12.7 | 6.4 |

N.A.: Not detected

According to Table 8, it can be known that in the LE/HPγCD formulations (Samples 2, 3, and 4) with different preparation ratios, the concentration of LE per unit HPγCD can be loaded in the range of 1.4-2.0 (μg/mg). However, when the water-soluble polymer, HPMC, and the formulation stabilizer, caffeine, (Samples 5, 6, 7 and 9) were added, the range of the amount of drug that can be loaded per unit of HPγCD was significantly increased to 12.7-16.4 (μg/mg). Namely, the solubility of LE in the formulation of water-soluble polymer, HPMC, and the formulation stabilizer, caffeine, was about 6.4-8.2 times that of Sample 4 (LE/HPγCD formula).

### Example 2

### Solubility Test for Axitinib

Effect of using caffeine as a stabilizer of a formulation on the solubility of the axitinib in the formulation containing axitinib/hydroxypropyl-γ- cyclodextrin (HPγCD)

### 1. Method

The samples were formulated and analyzed according to the formula shown in the following Table 9 and the methods described below at room temperature.

Hydroxypropyl-γ-cyclodextrin, a water-soluble polymer (hydroxypropyl methyl cellulose (HPMC)), and caffeine as a stabilizer of the formulation were dissolved in 3 mL of deionized water to form a solution.

Next, in an ultrasonic water bath environment, the above solution was slowly added to an acetic acid solution containing 4.05 mg axitinib (9 mg axitinib/mL) to form a mixture. After that, the mixture was lyophilized to remove the solvent therein and obtain a dried product.

Thereafter, the dried product was re-dissolved in 1 mL of deionized water to form a test sample, and the test sample solution was adjusted to pH 4.3 with 1 M NaOH. Next, the test sample was then filtered with a 0.22 μm pore size filter to remove the undissolved precipitate. Finally, the content of axitinib in the test sample was analyzed by high performance liquid chromatography (HPLC).

TABLE 9

| Sample | Axitinib | HPγCD | HPMC | Caffeine |
|---|---|---|---|---|
| number | (mg) | (mg) | (mg) | (mg) |
| 1 | 4.05 | — | — | — |
| 2 | 4.05 | 130.83 | — | — |
| 3 | 4.05 | — | 5 | — |
| 4 | 4.05 | — | — | 30 |
| 5 | 4.05 | 130.83 | 5 | — |
| 6 | 4.05 | 130.83 | — | 30 |
| 7 | 4.05 | — | 5 | 30 |
| 8 | 4.05 | 130.83 | 5 | 30 |

—: No addition

### 2. Results

The solubility of axitinib of each sample and the degree of solubility improvement compared to axitinib/HPγCD are as shown in Table 10 below.

TABLE 10

| | | The amount of | Fold of |
|---|---|---|---|
| | | axitinib that can be | solubility |
| | Axitinib | loaded per unit of | relative to |
| Sample | content | HP γCD | axitinib/ |
| number | (μg/mL) | (μg/mg) | HPγCD |
| 1 | <LOQ (4 μg/mL) | N.A. | N.A. |
| 2 | 105.55 | 8.07 | 1 |
| 3 | 14.55 | N.A. | N.A. |
| 4 | 55.30 | N.A. | N.A. |
| 5 | 375.43 | 2.87 | 3.56 |
| 6 | 226.49 | 1.73 | 2.15 |
| 7 | 270.23 | N.A. | N.A. |
| 8 | 1833.53 | 1.40 | 17.37 |

N.A.: Not detected
LOQ: Limit of quantification

According to Table 10 above, it can be known that the water solubility of axitinib (Sample 1) is extremely low, which is lower than the minimum drug content analytical limit (4 μg/mL). Based on the test result for sample 2, it can be found that when axitinib is mixed with HPγCD, the axitinib content in the solution can be effectively increased to 105.55 μg/mL. Moreover, the test results for the Samples 5 and 6 show that when HPγCD is combined with the water-soluble polymer, HPMC, or the formulation stabilizer, caffeine, the solubility of axitinib can be further improved to 2 to 3.6 times that of the axitinib/HPγCD formulation (Sample 2). Furthermore, if the axitinib/HPγCD formula is combined with the water-soluble polymer, HPMC, and the formulation stabilizer, caffeine, at the same time (Sample 8), the amount of axitinib dissolved can be greatly increased to 1833.53 μg/mL, which about 17.4 times that of the axitinib/HPγCD formula (Sample 2).

### Example 3

### Solubility Test for Type I Tyrosine Kinase Inhibitor

### Solubility Test for Cabozantinib

Effect of composition containing hydroxypropyl-P-cyclodextrin (HIVCD), hydroxypropyl methyl cellulose (HPMC) and caffeine in different proportions on the solubility of the cabozantinib

### 1. Method

The samples were formulated and analyzed according to the formula shown in the following Table 11 and the methods described below at room temperature.

(1) Powder of hydroxypropyl-β-cyclodextrin (HPβCD) was weighted and dissolved in deionized water to form a 200 mg/mL HPβCD aqueous solution. After that, the 200 mg/mL HPβCD aqueous solution was further diluted with different amounts of deionized water to respectively form 20-170 mg/mL HPβCD aqueous solutions.

(2) Powder of hydroxypropyl methyl cellulose (HPMC) was weighted and dissolved in deionized water to form a 5 mg/mL HPMC aqueous solution.

(3) Powder of caffeine was weighted and dissolved in deionized water to form a 40 mg/mL caffeine aqueous solution. After that, the 40 mg/mL caffeine aqueous solution was further diluted with different amounts of deionized water to respectively form 0.00675-36 mg/mL caffeine aqueous solutions.

(4) Cabozantinib was dissolved in dimethyl sulfoxide (DMSO) to form a 40 mg/mLcabozantinib solution.

(5) Based on Table 11, 1 mL HPβCD aqueous solution, 1 mL HPMC aqueous solution and 1 mL caffeine aqueous solution were added to a 20 mL vial and then 0.1 mL cabozantinib solution was further added therein to form a mixed solution with 4 mg content of cabozantinib. After that, the sample in the vial was sonicated in a water bath for 30 minutes.

(6) Next, the mixed solution was lyophilized to form a lyophilizate.

(7) The lyophilizate was re-dissolved in 1 mL deionized water and sonicated for 30 minutes to form a test sample.

(8) After standing for 24 hours, the test sample was filtered with a 0.22 μm pore-sized filter to remove the undissolved precipitate.

(9) Finally, the content of cabozantinib in the test sample was analyzed by high performance liquid chromatography (HPLC).

TABLE 11

| | | | | | HPβCD | HPMC | Caffeine | Cabozantinib |
|---|---|---|---|---|---|---|---|---|
| Sample | Cabozantinib | HPβCD | HPMC | Caffeine | fraction | fraction | fraction | concentration |
| number | (mg/mL) | (mg/mL) | (mg/mL) | (mg/mL) | (wt %) | (wt %) | (wt %) | (μg/mL) |
| 1 | 4.00 | — | — | — | — | — | — | 0.00 (<LOQ) |
| 2 | 4.00 | 200.00 | — | — | — | — | — | 103.30 |
| 3 | 4.00 | 200.00 | 5.00 | — | — | — | — | 440.20 |
| 4 | 4.00 | 200.00 | 5.00 | 0.01 | 97.56 | 2.44 | 0.0033 | 498.26 |
| 5 | 4.00 | 200.00 | 5.00 | 0.04 | 97.54 | 2.44 | 0.02 | 471.17 |
| 6 | 4.00 | 200.00 | 5.00 | 0.08 | 97.52 | 2.44 | 0.04 | 501.50 |
| 7 | 4.00 | 200.00 | 5.00 | 0.10 | 97.51 | 2.44 | 0.05 | 909.96 |
| 8 | 4.00 | 200.00 | 5.00 | 0.20 | 97.47 | 2.44 | 0.10 | 1499.03 |
| 9 | 4.00 | 200.00 | 5.00 | 0.40 | 97.37 | 2.43 | 0.19 | 1327.74 |
| 10 | 4.00 | 200.00 | 5.00 | 0.80 | 97.18 | 2.43 | 0.39 | 1376.29 |
| 11 | 4.00 | 200.00 | 5.00 | 1.60 | 96.81 | 2.42 | 0.77 | 1744.13 |
| 12 | 4.00 | 200.00 | 5.00 | 3.20 | 96.06 | 2.40 | 1.54 | 3000.17 |
| 13 | 4.00 | 200.00 | 5.00 | 5.00 | 95.24 | 2.38 | 2.38 | 2960.27 |
| 14 | 4.00 | 200.00 | 5.00 | 6.40 | 94.61 | 2.37 | 3.03 | 3259.41 |
| 15 | 4.00 | 200.00 | 5.00 | 7.50 | 94.12 | 2.35 | 3.53 | 3162.88 |
| 16 | 4.00 | 200.00 | 5.00 | 10.00 | 93.02 | 2.33 | 4.65 | 3024.41 |
| 17 | 4.00 | 200.00 | 5.00 | 12.80 | 91.83 | 2.30 | 5.88 | 3351.34 |
| 18 | 4.00 | 200.00 | 5.00 | 15.00 | 90.91 | 2.27 | 6.82 | 2914.96 |
| 19 | 4.00 | 200.00 | 5.00 | 20.00 | 88.89 | 2.22 | 8.89 | 3374.97 |
| 20 | 4.00 | 200.00 | 5.00 | 25.60 | 86.73 | 2.17 | 11.10 | 3182.55 |
| 21 | 4.00 | 200.00 | 5.00 | 30.00 | 85.11 | 2.13 | 12.77 | 3004.54 |
| 22 | 4.00 | 200.00 | 5.00 | 32.00 | 84.39 | 2.11 | 13.50 | 3132.19 |
| 23 | 4.00 | 200.00 | 5.00 | 33.75 | 83.77 | 2.09 | 14.14 | 3102.99 |
| 24 | 4.00 | 200.00 | 5.00 | 36.00 | 82.99 | 2.07 | 14.94 | 3232.01 |
| 25 | 4.00 | 200.00 | 5.00 | 40.00 | 81.63 | 2.04 | 16.33 | 3053.42 |
| 26 | 4.00 | 170.00 | 5.00 | 40.00 | 79.07 | 2.33 | 18.60 | 2988.12 |
| 27 | 4.00 | 145.00 | 5.00 | 40.00 | 76.32 | 2.63 | 21.05 | 3046.02 |
| 28 | 4.00 | 120.00 | 5.00 | 40.00 | 72.73 | 3.03 | 24.24 | 3121.94 |
| 29 | 4.00 | 95.00 | 5.00 | 40.00 | 67.86 | 3.57 | 28.57 | 3063.76 |
| 30 | 4.00 | 70.00 | 5.00 | 40.00 | 60.87 | 4.35 | 34.78 | 1254.19 |
| 31 | 4.00 | 45.00 | 5.00 | 40.00 | 50.00 | 5.56 | 44.44 | 447.07 |
| 32 | 4.00 | 20.00 | 5.00 | 40.00 | 30.77 | 7.69 | 61.54 | 216.10 |

—: No addition
LOQ: Limit of quantification

### 2. Results

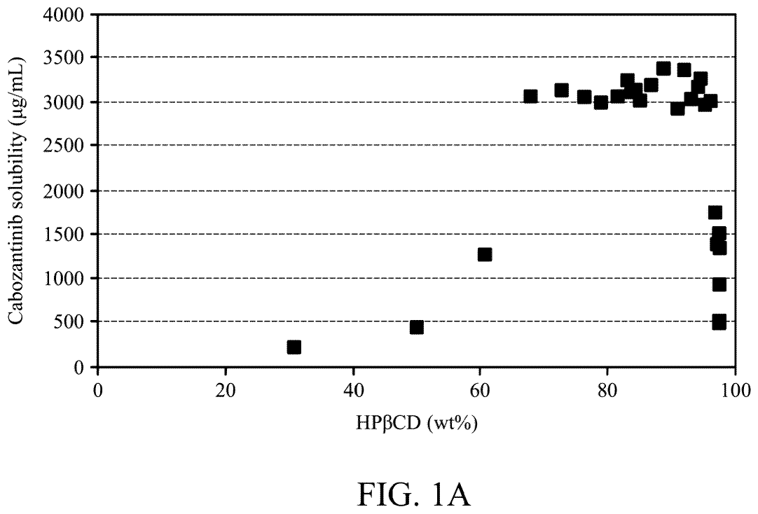

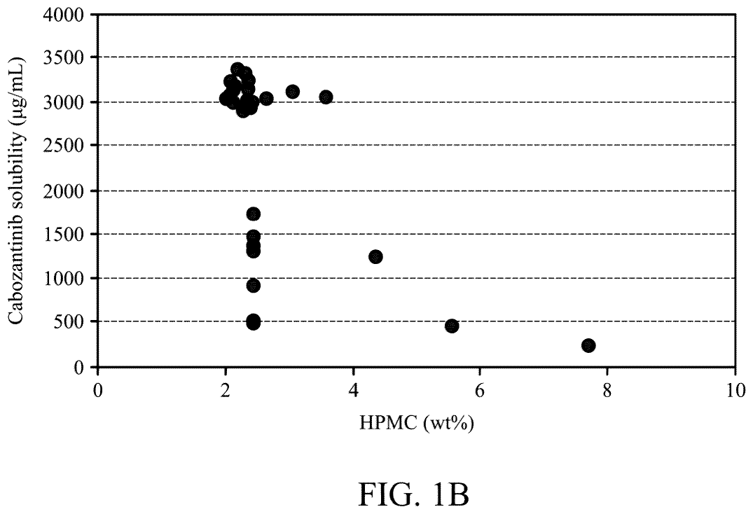

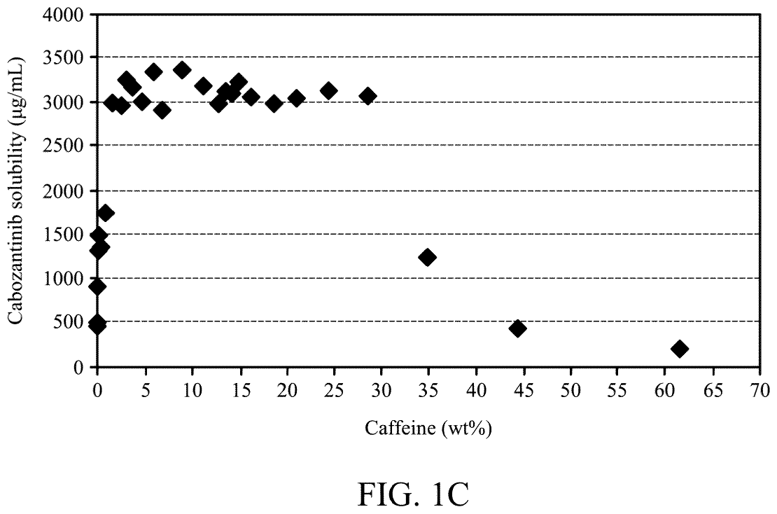

The solubility of cabozantinib of each sample is as shown in Table 11 above. Moreover, the effects of the respective contents of hydroxypropyl-β-cyclodextrin, hydroxypropyl methyl cellulose and caffeine in the composition on the solubility of cabozantinib in the formulation are shown in FIG. 1A, FIG. 1B and FIG. 1C.

Table 11 and FIG. 1A, FIG. 1B and FIG. 1C clearly show that the solubility of cabozantinib has significantly increased from Sample 11 to Sample 29 compared to other samples.

According to the results mentioned above, it is assumed that the solubility of cabozantinib can be significantly increased by a composition comprising about 60-97% by weight of hydroxypropyl-β-cyclodextrin, about 0.5-4% by weight of hydroxypropyl methyl cellulose and about 0.4-30% by weight of caffeine. Specifically, for improving the solubility of cabozantinib, the content of hydroxypropyl-γ-cyclodextrin in the composition has to be within a range of about 60-97% by weight, the content of hydroxypropyl methyl cellulose in the composition has to be within a range of about 0.5-4% by weight and the content of caffeine in the composition has to be within a range of about 0.4-30% by weight at the same time.

### Example 4

### Solubility Test for Type II Tyrosine Kinase Inhibitor

### Solubility Test for Axitinib

Effect of composition containing hydroxypropyl-γ-cyclodextrin (HPγCD), hydroxypropyl methyl cellulose (HPMC) and caffeine in different proportions on the solubility of the axitinib.

### 1. Method

The samples were formulated and analyzed according to the formula shown in the following Table 12 and the methods described below at room temperature.

(1) Powder of hydroxypropyl-γ-cyclodextrin (HPγCD) was weighted and dissolved in deionized water to form a 130 mg/mL HPγCD aqueous solution. After that, the 130 mg/mL HPγCD aqueous solution was further diluted with different amounts of deionized water to respectively form 20-120 mg/mL HPγCD aqueous solutions.

(2) Powder of hydroxypropyl methyl cellulose (HPMC) was weighted and dissolved in deionized water to form a 5 mg/mL HPMC aqueous solution.

(3) Powder of caffeine was weighted and dissolved in deionized water to form a 40 mg/mL caffeine aqueous solution. After that, the 40 mg/mL caffeine aqueous solution was further diluted with different amounts of deionized water to respectively form 0.00675-36 mg/mL caffeine aqueous solutions.

(4) Axitinib was dissolved in glacial acetic acid to form a 20 mg/mL axitinib solution.

(5) Based on Table 12, 1 mL HPγCD aqueous solution, 1 mL HPMC aqueous solution and 1 mL caffeine aqueous solution were added to a 20 mL vial and then 0.2 mL axitinib solution was further added therein to form a mixed solution with 4 mg content of axitinib. After that, the sample in the vial was sonicated in a water bath for 30 minutes.

(6) Next, the mixed solution was lyophilized to form a lyophilizate.

(7) The lyophilizate was re-dissolved in 1 mL deionized water and sonicated for 30 minutes to form a test sample.

(8) After standing for 24 hours, the test sample was filtered with a 0.22 μm pore-sized filter to remove the undissolved precipitate.

(9) Finally, the content of axitinib in the test sample was analyzed by high performance liquid chromatography (HPLC).

TABLE 12

| | | | | | HPγCD | HPMC | Caffeine | Axitinib |
|---|---|---|---|---|---|---|---|---|
| Sample | Axitinib | HPγCD | HPMC | Caffeine | fraction | fraction | fraction | concentration |
| number | (mg/mL) | (mg/mL) | (mg/mL) | (mg/mL) | (wt %) | (wt %) | (wt %) | (μg/mL) |
| 1 | 4 | — | — | — | — | — | — | 0 (<LOQ) |
| 2 | 4 | 130 | — | — | — | — | — | 124.5 |
| 3 | 4 | 130 | 5 | — | — | — | — | 409.593 |
| 4 | 4 | 130 | 5 | 0.00675 | 96.291 | 3.704 | 0.005 | 307.687 |
| 5 | 4 | 130 | 5 | 0.0405 | 96.267 | 3.702 | 0.03 | 370.762 |
| 6 | 4 | 130 | 5 | 0.081 | 96.239 | 3.701 | 0.06 | 409.194 |
| 7 | 4 | 130 | 5 | 0.1 | 96.225 | 3.701 | 0.074 | 322.518 |
| 8 | 4 | 130 | 5 | 0.2 | 96.154 | 3.698 | 0.148 | 390.851 |
| 9 | 4 | 130 | 5 | 0.4 | 96.012 | 3.693 | 0.295 | 982.876 |
| 10 | 4 | 130 | 5 | 0.8 | 95.729 | 3.682 | 0.589 | 1134.903 |
| 11 | 4 | 130 | 5 | 1.6 | 95.168 | 3.66 | 1.171 | 1493.487 |
| 12 | 4 | 130 | 5 | 3.2 | 94.067 | 3.618 | 2.315 | 1214.046 |
| 13 | 4 | 130 | 5 | 5 | 92.857 | 3.571 | 3.571 | 1684.869 |
| 14 | 4 | 130 | 5 | 6.4 | 91.938 | 3.536 | 4.526 | 1999.385 |
| 15 | 4 | 130 | 5 | 7.5 | 91.228 | 3.509 | 5.263 | 1544.508 |
| 16 | 4 | 130 | 5 | 10 | 89.655 | 3.448 | 6.897 | 1778.793 |
| 17 | 4 | 130 | 5 | 12.8 | 87.957 | 3.383 | 8.66 | 1308.953 |
| 18 | 4 | 130 | 5 | 15 | 86.667 | 3.333 | 10 | 1533.188 |
| 19 | 4 | 130 | 5 | 20 | 83.871 | 3.226 | 12.903 | 2108.915 |
| 20 | 4 | 130 | 5 | 25.6 | 80.946 | 3.113 | 15.94 | 1303.805 |
| 21 | 4 | 130 | 5 | 30 | 78.788 | 3.03 | 18.182 | 1612.556 |
| 22 | 4 | 130 | 5 | 32 | 77.844 | 2.994 | 19.162 | 1152.392 |
| 23 | 4 | 130 | 5 | 33.75 | 77.037 | 2.963 | 20 | 1608.967 |
| 24 | 4 | 130 | 5 | 36 | 76.023 | 2.924 | 21.053 | 1434.823 |
| 25 | 4 | 130 | 5 | 40 | 74.286 | 2.857 | 22.857 | 1327.753 |
| 26 | 4 | 80 | 5 | 40 | 64 | 4 | 32 | 326.372 |
| 27 | 4 | 60 | 5 | 40 | 57.143 | 4.762 | 38.095 | 344.08 |
| 28 | 4 | 40 | 5 | 40 | 47.059 | 5.882 | 47.059 | 213.143 |
| 29 | 4 | 30 | 5 | 40 | 40 | 6.667 | 53.333 | 183.915 |
| 30 | 4 | 20 | 5 | 40 | 30.769 | 7.692 | 61.538 | 211.901 |

—: No addition
LOQ: Limit of quantification

### 2. Results

The solubility of axitinib of each sample is as shown in Table 12 above.

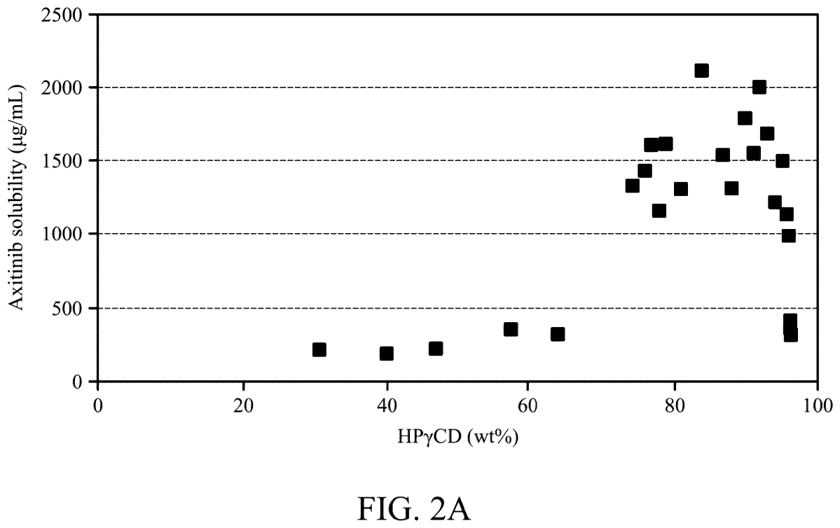

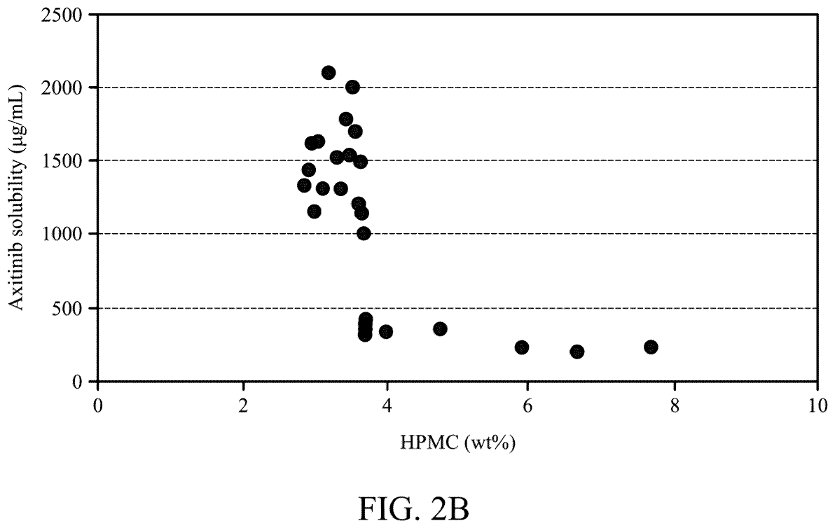

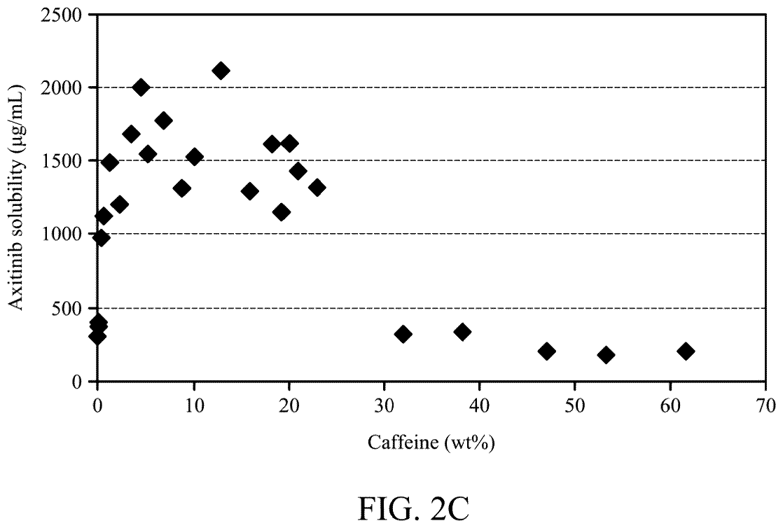

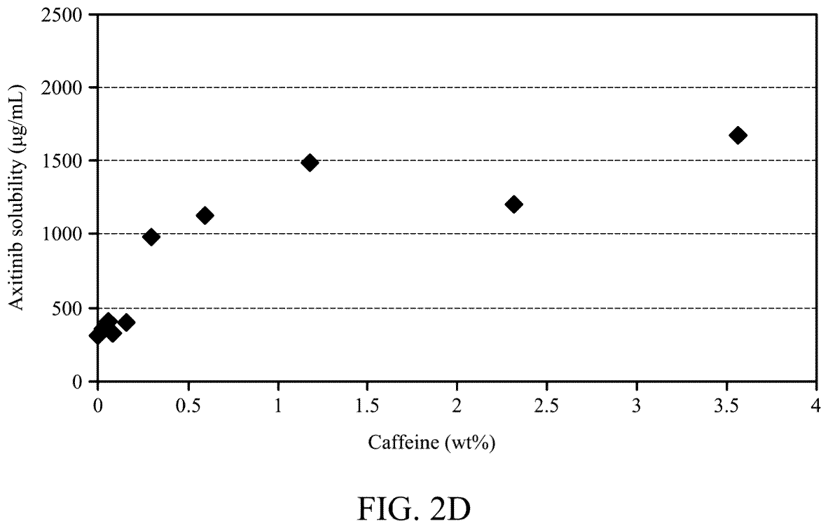

Moreover, the effects of the respective contents of hydroxypropyl-γ-cyclodextrin, hydroxypropyl methyl cellulose and caffeine in the composition on the solubility of axitinib in the formulation are shown in FIG. 2A, FIG. 2B and FIGS. 2C and D.

Table 12 and FIG. 2A, FIG. 2B and FIGS. 2C and 2D clearly show that the solubility of axitinib has significantly increased from Sample 10 to Sample 25 compared to other samples.

According to the results mentioned above, it is assumed that the solubility of axitinib can be significantly increased by a composition comprising about 60-97% by weight of hydroxypropyl-γ-cyclodextrin, about 0.5-4% by weight of hydroxypropyl methyl cellulose and about 0.4-30% by weight of caffeine. Specifically, for improving the solubility of axitinib, the content of hydroxypropyl-γ-cyclodextrin in the composition has to be within a range of about 60-97% by weight, the content of hydroxypropyl methyl cellulose in the composition has to be within a range of about 0.5-4% by weight and the content of caffeine in the composition has to be within a range of about 0.4-30% by weight at the same time.

### Example 5

### Solubility Tests for Type III Tyrosine Kinase Inhibitor

### Solubility Test for Binimetinib

Effect of composition containing hydroxypropyl-γ-cyclodextrin (HPγCD), hydroxypropyl methyl cellulose (HPMC) and caffeine in different proportions on the solubility of the binimetinib.

### 1. Method

The samples were formulated and analyzed according to the formula shown in the following Table 13 and the methods described below at room temperature.

(1) Powder of hydroxypropyl-γ-cyclodextrin (HPγCD) was weighted and dissolved in deionized water to form a 80 mg/mL HPγCD aqueous solution. After that, the 80 mg/mL HPγCD aqueous solution was further diluted with different amounts of deionized water to respectively form 10-70 mg/mL HPγCD aqueous solutions.

(2) Powder of hydroxypropyl methyl cellulose (HPMC) was weighted and dissolved in deionized water to form a 3 mg/mL HPMC aqueous solution.

(3) Powder of caffeine was weighted and dissolved in deionized water to form a 40 mg/mL caffeine aqueous solution. After that, the 40 mg/mL caffeine aqueous solution was further diluted with different amounts of deionized water to respectively form 0.00675-36 mg/mL caffeine aqueous solutions.

(4) Binimetinib was dissolved in dimethyl sulfoxide (DMSO) to form a 40 mg/mL binimetinib solution.

(5) Based on Table 13, 1 mL HPγCD aqueous solution, 1 mL HPMC aqueous solution and 1 mL caffeine aqueous solution were added to a 20 mL vial and then 0.1 mL binimetinib solution was further added therein to form a mixed solution with 4 mg content of binimetinib. After that, the sample in the vial was sonicated in a water bath for 30 minutes.

(6) Next, the mixed solution was lyophilized to form a lyophilizate.

(7) The lyophilizate was re-dissolved in 1 mL deionized water and sonicated for 30 minutes to form a test sample.

(8) After standing for 24 hours, the test sample was filtered with a 0.22 μm pore-sized filter to remove the undissolved precipitate.

(9) Finally, the content of binimetinib in the test sample was analyzed by high performance liquid chromatography (HPLC).

TABLE 13

| | | | | | HPγCD | HPMC | Caffeine | Binimetinib |
|---|---|---|---|---|---|---|---|---|
| Sample | Binimetinib | HPγCD | HPMC | Caffeine | fraction | fraction | fraction | concentration |
| number | (mg/mL) | (mg/mL) | (mg/mL) | (mg/mL) | (wt %) | (wt %) | (wt %) | (μg/mL) |
| 1 | 4 | — | — | — | — | — | — | 40 |
| 2 | 4 | 80 | — | — | — | — | — | 550 |
| 3 | 4 | 80 | 3 | — | — | — | — | 1910 |
| 4 | 4 | 80 | 3 | 0.8 | 95.465 | 3.580 | 0.955 | 3610 |
| 5 | 4 | 80 | 3 | 1.6 | 94.563 | 3.546 | 1.891 | 3660 |
| 6 | 4 | 80 | 3 | 3.2 | 92.807 | 3.480 | 3.712 | 3590 |
| 7 | 4 | 80 | 3 | 5 | 90.909 | 3.409 | 5.682 | 3670 |
| 8 | 4 | 80 | 3 | 6.4 | 89.485 | 3.356 | 7.159 | 3630 |
| 9 | 4 | 80 | 3 | 7.5 | 88.398 | 3.315 | 8.287 | 3600 |
| 10 | 4 | 80 | 3 | 10 | 86.022 | 3.226 | 10.753 | 3680 |
| 11 | 4 | 80 | 3 | 12.8 | 83.507 | 3.132 | 13.361 | 3600 |
| 12 | 4 | 80 | 3 | 15 | 81.633 | 3.061 | 15.306 | 3640 |
| 13 | 4 | 80 | 3 | 20 | 77.670 | 2.913 | 19.417 | 3560 |
| 14 | 4 | 80 | 3 | 25.6 | 73.665 | 2.762 | 23.573 | 3600 |
| 15 | 4 | 80 | 3 | 30 | 70.796 | 2.655 | 26.549 | 3610 |
| 16 | 4 | 80 | 3 | 32 | 69.565 | 2.609 | 27.826 | 3610 |
| 17 | 4 | 80 | 3 | 33.75 | 68.522 | 2.570 | 28.908 | 3230 |
| 18 | 4 | 20 | 3 | 40 | 31.746 | 4.7619 | 63.492 | 1580 |
| 19 | 4 | 10 | 3 | 40 | 18.868 | 5.660 | 75.472 | 670 |

—: No addition

### 2. Results

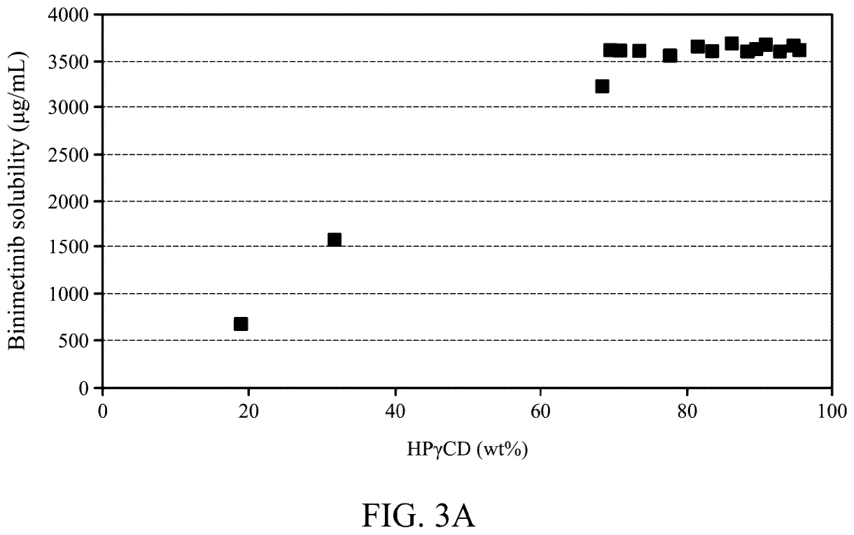

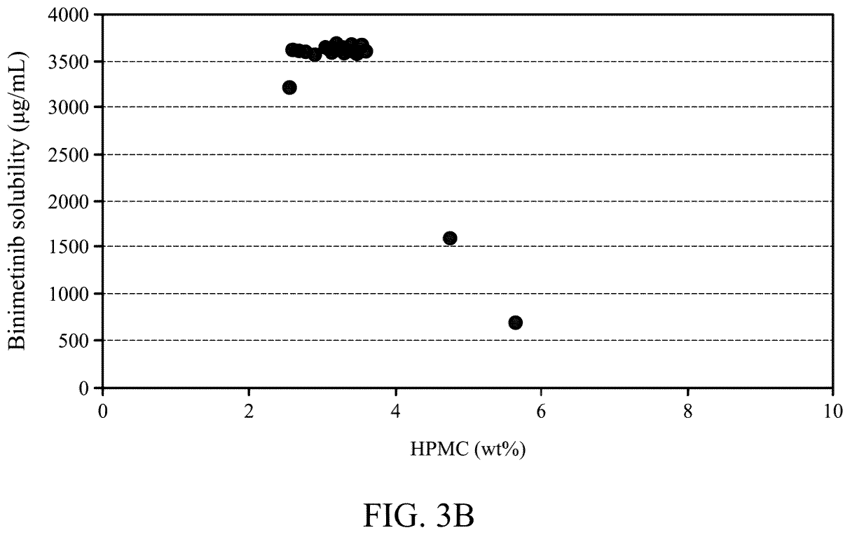

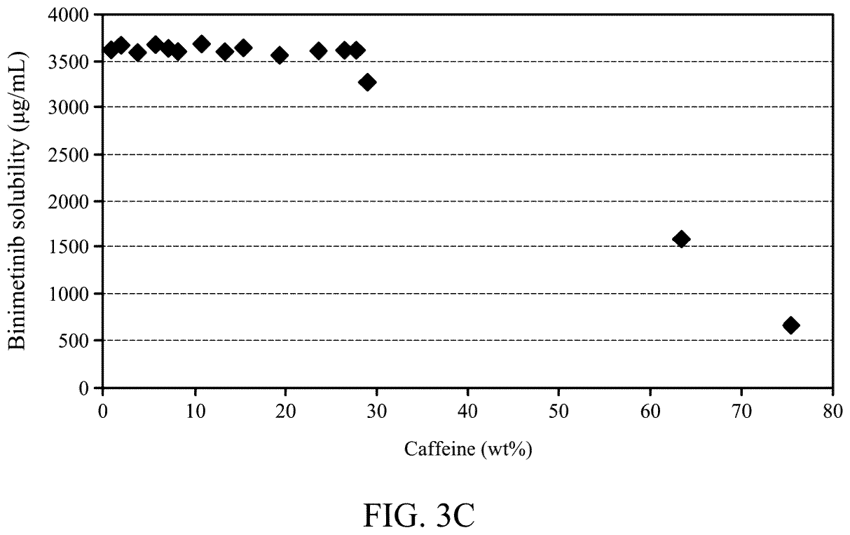

The solubility of binimetinib of each sample is as shown in Table 15 above. Moreover, the effects of the respective contents of hydroxypropyl-γ-cyclodextrin, hydroxypropyl methyl cellulose and caffeine in the composition on the solubility of binimetinib in the formulation are shown in FIG. 3A, FIG. 3B and FIG. 3C.

Table 13 and FIG. 3A, FIG. 3B and FIG. 3C clearly show that the solubility of binimetinib has significantly increased from Sample 4 to Sample 17 compared to other samples.

According to the results mentioned above, it is assumed that the solubility of binimetinib can be significantly increased by a composition comprising about 60-97% by weight of hydroxypropyl-γ-cyclodextrin, about 0.5-4% by weight of hydroxypropyl methyl cellulose and about 0.4-30% by weight of caffeine. Specifically, for improving the solubility of binimetinib, the content of hydroxypropyl-γ-cyclodextrin in the composition has to be within a range of about 60-97% by weight, the content of hydroxypropyl methyl cellulose in the composition has to be within a range of about 0.5-4% by weight and the content of caffeine in the composition has to be within a range of about 0.4-30% by weight at the same time.

### Example 6

### Molecular Dynamic Simulation of Loteprednol Etabonate/Cyclodextrin/Glutathione Complexation

In order to simulate the binding energy and structures of the complexes correctly, the structure of each component including carrier, drug, destabilizer, and stabilizer was optimized first. All geometry optimizations of the component molecules in water (modeled by PCM) were performed using gradient-corrected hybrid density functional theory (DFT) within the Gaussian 16 suite of programs (Frisch, M. J., et al.) on a PC cluster at the National Center for High-Performance Computing, Taiwan. The B3LYP density functional, Becke's three-parameter exchange functional (D. J. Gaussian 16, Wallingford, CT, 2016) and Lee-Yang-Parr gradient-corrected correlation functional (Becke, A. D., Density-Functional Thermochemistry. III. The Role of Exact Exchange. J. Chem. Phys. 1993, 98, 5648.) were utilized. The moderate-sized 6-31G (d,p) basis set (Lee, C.; Yang, W.; Parr, R. G., Development of the Colle-Salvetti Correlation-Energy Formula Into a Functional of the Electron Density. Physical Review B 1988, 37, 785.) was used. The calculated stable structures were examined in terms of vibrational frequency calculations. The optimized structures are used for the following docking studies.

### 1. Structure of hydroxypropyl-γ- cyclodextrin (HPγCD)

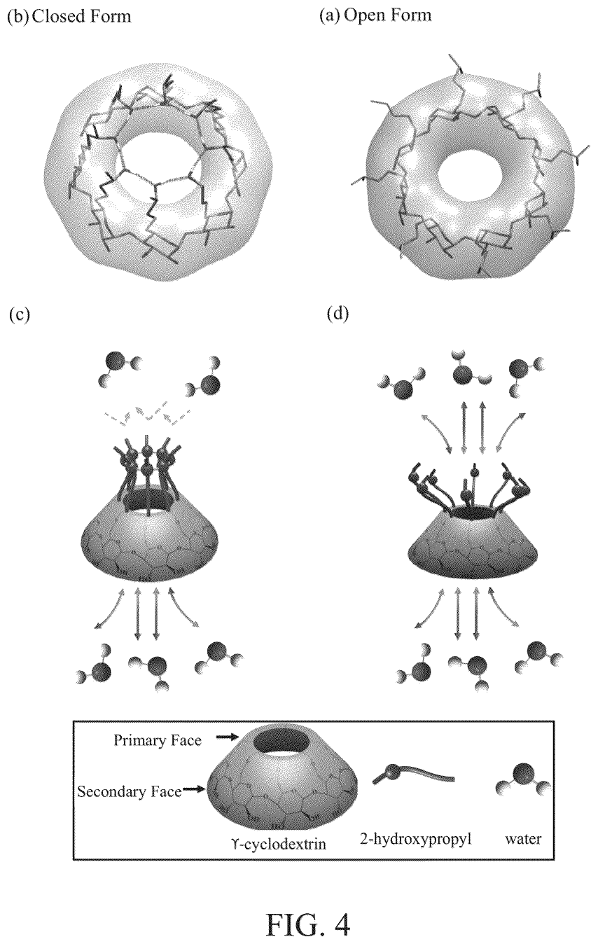

Hydroxypropyl-γ-cyclodextrin (HPγCD), served as the carrier, has two distinct conformations. The hydroxypropyl groups of HPγCD can aggregate together, thereby closing the primary face of HPγCD. The other conformation of HPγCD has its hydroxypropyl groups dispersed in water, thereby opening the primary face of HPγCD. FIG. 4 shows the optimized structures of HPγCD. The closed-form of HPγCD has its hydroxypropyl groups formed hydrogen-bond networks (FIG. 4 (a)), which is 34.7 kcal/mol more stable than the corresponding open form. Therefore, the closed form of HPγCD does not allow the water molecules passing through freely (see FIG. 4 (c)). In contrast, the water molecules can pass through the open form of HPγCD easily (see FIG. 4 (d)). Accordingly, the closed-form of HPγCD was employed for the following studies.

### 2. Docking Simulations of Inclusion Complexes

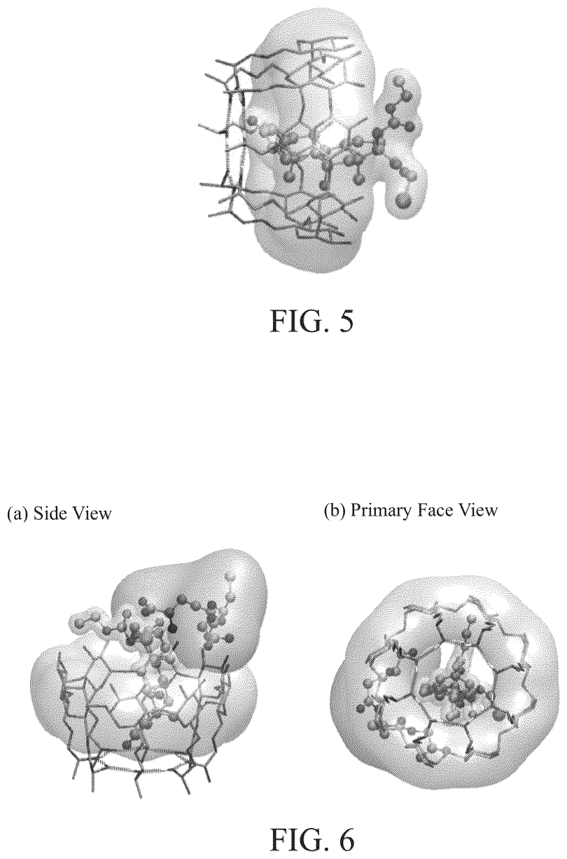

AutoDock Vina 1.125 was employed to screen the stable structures of the inclusion complexes. Table 14 lists the top 5 docking results of [LE@HPγCD] inclusion complex. The initial structures of LE and HPγCD used for docking simulations are obtained from DFT calculations mentioned above (see previous section). It is observed that the top 5 docking results of [LE@HPγCD] inclusion complex have similar binding affinity of -8.9 kcal/mol, indicating that the LE does not form specific interactions with the symmetric HPγCD. The top 1 docking structure of [LE@HPγCD] inclusion complex is as shown in FIG. 5. In the structure of [LE@HPγCD] inclusion complex, the polar moiety of LE is located outside the secondary face of HPγCD as well as the hydrophobic moiety is located inside the cavity of HPγCD.

TABLE 14
The top 5 docking results of [LE@HPγCD] inclusion complex.

| Mode | Affinity (kcal/mol) |
|---|---|
| 1 | −8.9 |
| 2 | −8.9 |
| 3 | −8.9 |
| 4 | −8.9 |
| 5 | −8.4 |

Table 15 lists the top 5 docking results of the GSH-[LE@HPγCD] inclusion complex. The GSH is located on the secondary face of HPγCD. The top 1 docking structure of GSH-[LE@HPγCD] inclusion complex is as shown in FIG. 6. In this structure, the GSH can prevent the LE to interact with water molecules directly.

TABLE 15
The top 5 docking results of GSH-[LE@HPγCD]
inclusion complex.

| Mode | Affinity (kcal/mol) | GSH Location |
|---|---|---|
| 1 | −3.9 | Secondary Face |
| 2 | −3.8 | Secondary Face |
| 3 | −3.8 | Secondary Face |
| 4 | −3.7 | Secondary Face |
| 5 | −3.6 | Primary Face |

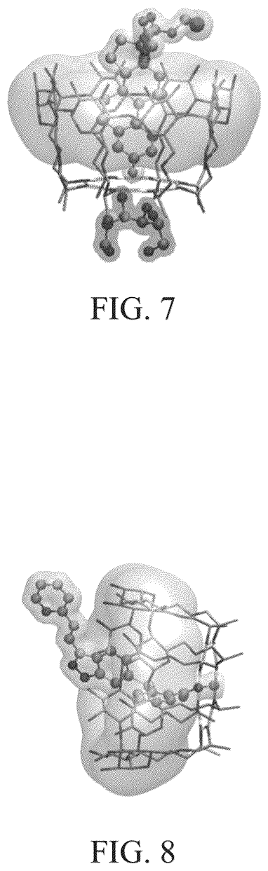

Table 16 lists the top 5 docking results of mannitol-[LE@HPγCD] inclusion complex. The highly water-soluble mannitol served as a negative control is located on the primary face of HPγCD. The top 1 docking structure of mannitol-[LE@HPγCD] inclusion complex is as shown in FIG. 7. In this structure, the polar mannitol can form hydrogen-bonds with the hydroxypropyl groups of HPγCD, which might partially destroy the hydrogen-bond networks of hydroxypropyl groups.

TABLE 16
Top 5 docking results of mannitol-[LE@HPγCD]
inclusion complex.

| Mode | Affinity (kcal/mol) |
|---|---|
| 1 | −3.4 |
| 2 | −3.3 |
| 3 | −3.3 |
| 4 | −3.3 |
| 5 | −3.2 |

Table 17 lists the top 5 docking results of [axitinib@HPγCD] inclusion complex. The initial structures of axitinib and HPγCD used for docking simulations are obtained from DFT calculations mentioned above. It is observed that the top 5 docking results of [axitinib@HPγCD] inclusion complex have similar binding affinity of -8.6 kcal/mol, indicating that the axitinib does not form specific interactions with the symmetric HPγCD. The top 1 docking structure of [axitinib@HPγCD] inclusion complex is as shown in FIG. 8. In the structure of [axitinib@HPγCD] inclusion complex, the polar moiety of axitinib is located outside the primary face of HPγCD as well as the hydrophobic moiety is located inside the cavity of HPγCD.

TABLE 17
The top 5 docking results of [axitinib@HPγCD]
inclusion complex.

| Mode | Affinity (kcal/mol) |
|---|---|
| 1 | −8.6 |
| 2 | −8.6 |
| 3 | −8.6 |
| 4 | −8.6 |
| 5 | −8.5 |

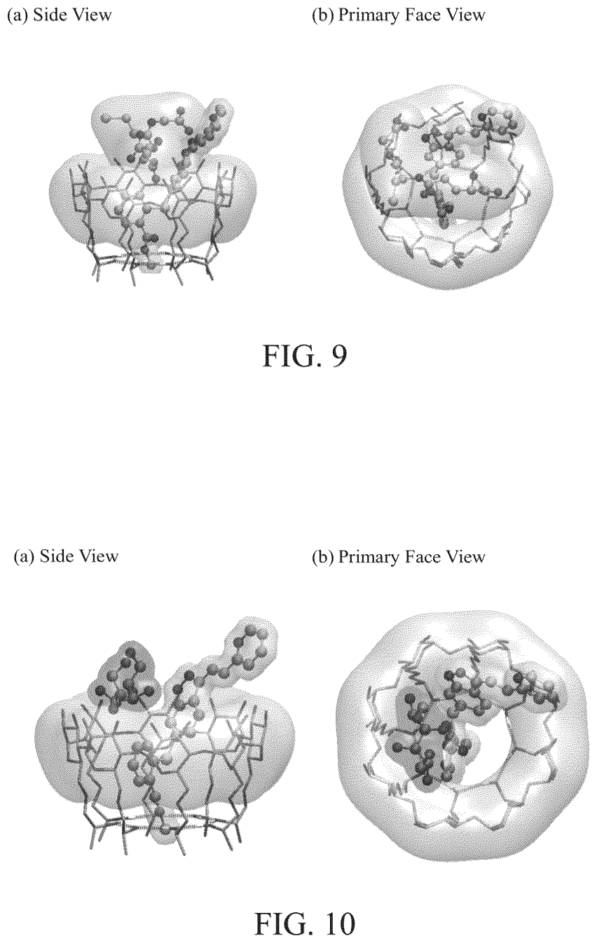

Table 18 lists the top 5 docking results of the GSH-[axitinib@HPγCD] inclusion complex. The GSH is located on the secondary face of HPγCD. The top 1 docking structure of GSH-[axitinib@HPγCD] inclusion complex is as shown in FIG. 9. In this structure, the stabilizer (GSH) can prevent the axitinib to interact with water molecules directly.

TABLE 18
Top 5 docking results of GSH-[axitinib@HPγCD]
inclusion complex

| Mode | Affinity (kcal/mol) | GSH Location |
|---|---|---|
| 1 | −4.9 | Secondary Face |
| 2 | −4.8 | Secondary Face |
| 3 | −4.8 | Secondary Face |
| 4 | −4.6 | Secondary Face |
| 5 | −2.6 | Primary Face |

Table 19 lists the top 5 docking results of mannitol-[axitinib@HPγCD] inclusion complex. The mannitol is located on the secondary face of HPγCD. The top 1 docking structure of mannitol-[axitinib@HPγCD] inclusion complex is as shown in FIG. 10.

TABLE 19
Top 5 docking results of mannitol-[axitinib@HPγCD]
inclusion complex.

| Mode | Affinity (kcal/mol) |
|---|---|
| 1 | −3.5 |
| 2 | −3.1 |
| 3 | −3.1 |
| 4 | −3.1 |
| 5 | −3.0 |

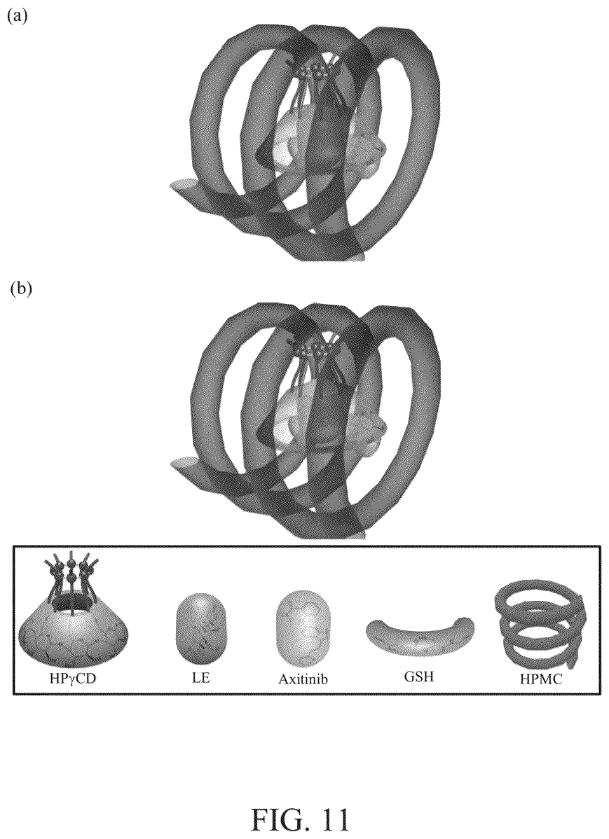

FIG. 11 illustrates the possible structures of hydroxypropyl methylcellulose GSH-[LE@HPγCD] and hydroxypropyl methylcellulose GSH-[axitinib@HPγCD] conjugates. The entanglement of HPMC with the inclusion complex can stabilize the [drug@HPγCD] inclusion complex.

### 3. Binding Energy of Inclusion Complexes

The binding energies of complexes were calculated with the basis set superposition error (BSSE) correction. The top 1 structure of complex from the docking calculations is used as the initial structure for further geometry optimization by B3LYP/6-31G(d) method. The vibrational frequencies of optimized complexes are further calculated to examine whether it is a stationary point or not. After all positive vibrational frequencies are obtained for the optimized complexes, the binding energy (AF) of complex is calculated by the following formula:

ΔE=E_{complex}−(E_{ligand}+E_{HPγCD})

Table 20 lists the binding energy of complexes. The smaller binding energy (-5.78 kcal/mol) of [drug@HPγCD] complex will allow the drugs to be released easily for performing its activity when the complex arrived at the target. Interestingly, the GSH has larger binding energy than mannitol. The rich H-bond donors and acceptors of mannitol make the mannitol highly water soluble (1.19 mol/L at 25° C.). The water solubility of GSH at 25° C. is 0.95 mol/L only.

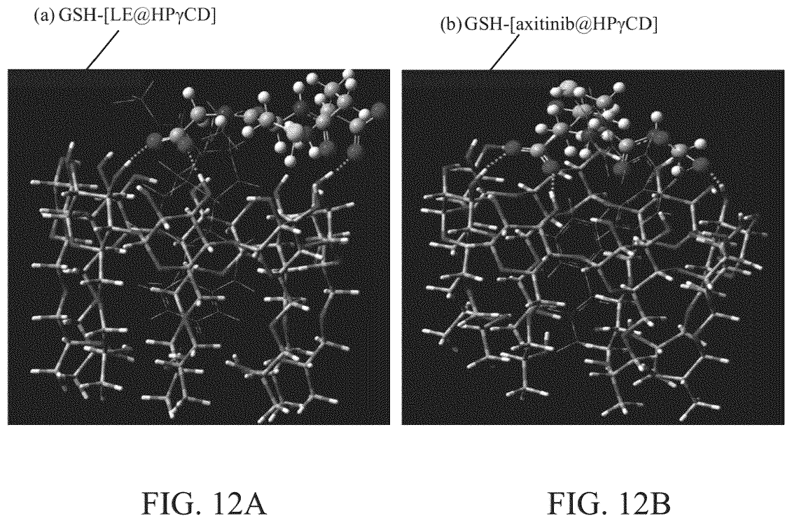

Table 21 lists the relative energies (calculated at B3LYP/6-31G(d) level) of GSH-[axitinb@HPγCD] structures with the GHS located at the primary and secondary faces, respectively. Based on Table 21, it can be known that the GSH-[axitinb@HPγCD] structure with the GHS located at the secondary face is 9 kcal/mol more stable than the one with the GHS located at the primary face. FIGS. 12A and 12B respectively shows the 3D structures of GSH-[LE@HPγCD] and GSH-[axitinib@HPγCD] inclusion complexes highlighting the H-bond interactions between the GSH and the secondary face of HPγCD.

TABLE 20
The binding energies (kcal/mol) of inclusion complexes calculated at B3LYP/6-31G(d) level

| System 1 | LE vs HPγCD | GSH vs [LE@HPγCD] | Mannitol vs [LE@HPγCD] |
|---|---|---|---|
| Binding Energy | −5.97 | −74.37 | −40.64 |
| (BSSE correction) | | | |
| System 2 | axitinib vs HPγCD | GSH vs [axitinb@HPγCD] | Mannitol vs [axitinb@HPγCD] |
| Binding Energy | −8.91 | −76.31 | −29.67 |
| BSSE correction) | | | |

TABLE 21
The relative energies (kcal/mol) of GSH-[axitinb@HPγCD]
structures with GSH located at the primary and secondary
faces calculated at B3LYP/6-31G(d) level.

| | Secondary Face | Primary Face |
|---|---|---|
| Relative Energy | 0.00 | 9.01 |

### Example 7

### Animal Experiment

### Example 7-1

### Determination of the Exposure Amount of LOTEPREDNOL ETABONATE (LE)

### 1. Sample Preparation

The samples were prepared according to the formula shown in the following Table 22 at room temperature. The sample preparation method is analogous to the foregoing Example 1-4. In the Sample 2 of this example, Tween 80 was further added to aggregate the sample to form microparticles, and the formed microparticles have an average particle diameter of about 500 nm to 100 μm.

TABLE 22

| | | | | | | | LE |
|---|---|---|---|---|---|---|---|
| | | LE | HPγCD | Caffeine | HPMC | Tween 80 | content |
| Number | Sample | (mg) | (mg) | (mg) | (mg) | (mg) | (μg/mL) |
| 1 | HPC8C15LH | 2 | 204 | 15 | 10 | — | 1710.0 |
| 2 | HPC8C15LH-TW80 | 2 | 204 | 15 | 10 | 10 | 1686.6 |

—: No addition

### 2. Method for Determining Exposure Amount

Male New Zealand White rabbits with a body weight of about 2-3 kg were weighed and recorded before the experiment. The rabbit was placed in a Baoding rack. After the rabbit was in a stable state, the lower eyelids were opened to expose the conjunctival sacs. Then, a 35 μL eyedrop formula was taken by a pipetman and dropped into the conjunctival sacs of the right and left eyes of the white rabbit, separately. After that, the eyelids were closed and gently rubbed so that the eye drops can moisten the entire surface of the eyes.

At 0.5, 1, and 3 hours after administration, the white rabbits were sacrificed with CO₂. Next, the left and right eyeballs were washed with PBS solution, first, and then the left and right eyeballs were taken out. After taking out the left and right eyeballs, the taken eyeballs of the white rabbits were washed again with PBS solution, and the excess PBS solution was removed with a dust-free paper.

A 25 G syringe was used to puncture the posterior cornea to draw the aqueous humor (AH) out, and the aqueous humor was then placed in a 1.5 mL centrifuge tube. Next, the eyeball tissue and the centrifuge tube containing the aqueous humor were placed in liquid nitrogen for about 2 minutes to be rapidly frozen, and then stored in a −80° C. refrigerator for subsequent analysis for a drug content of a sample. 20 μL of aqueous humor sample or the standard for the drug with different concentrations was respectively added to a 1.5 mL centrifuge tube. 180 μL of ACN (acetonitrile) containing 0.1% TFA (Trifluoroacetic acid) was added to each centrifuge tube and mixed well. The centrifuge tube was placed in a centrifuge and centrifuged at 15,000 rpm for 10 minutes. The centrifuged sample was subjected to liquid chromatography-tandem mass spectrometry (LC-MS/MS) for quantitative analysis of drug concentration in the aqueous humor sample (LLOQ: 0.1 ng/mL), and the pharmacokinetic parameters were calculated (time required for reaching the highest blood drug concentration Tₘₐₓ, the highest blood drug concentration Cₘₐₓ, the area under blood drug concentration-time curve, AUC (Area under curve), etc.) to evaluate the ability of increasing corneal penetration of the formulations of the drug.

### 3. Results

### 2-1 Determination of the Exposure Amount of Loteprednol Etabonate (LE)

The formulation of the present disclosure, HPC8C15LH, HPC8C15LH-TW80, or Lotemax (a commercial product of loteprednol etabonate (LE)) (Bausch & Lomb, Inc.) was respectively administered to the white rabbits by the aforementioned determination methods, and the results are as shown in the following Table 23.

TABLE 23

| | | | |
|---|---|---|---|
| | Tₘₐₓ | Cₘₐₓ | AUC_{0-3 hour} |
| PK parameter | (hour) | (ng/mL) | (hour × ng/mL) |

Aqueous humor

| | | | |
|---|---|---|---|
| HPC8C15LH | 0.5 | 14.3 ± 1.8 | 17.7 ± 1.7 |
| HPC8C15LH-TW80 | 0.5 | 157.8 ± 37.2 | 145.3 ± 25.8 |
| Lotemax | 0.5 | 6 ± 1 | 14 ± 2 |

Iris/Ciliary body

| | | | |
|---|---|---|---|
| HPC8C15LH | 0.5 | 471 ± 86 | 493 ± 43 |
| HPC8C15LH-TW80 | 0.5 | 95 ± 26 | 105 ± 23 |
| Lotemax | 0.5 | 49 ± 6 | 91 ± 5 |

According to Table 23, it can be known that the formulation of the present disclosure can enhance the penetration ability of the drug into the anterior chamber of the eye, and can increase the AUC of the drug in the aqueous humor by more than 10 times.

### Example 7-2

### Determination of the Exposure Amount of Axitinib

### 1. Sample Preparation

Samples were prepared according to the formulations as shown in Table 24 below at room temperature. The sample preparation method is analogous to the foregoing Example 2.

TABLE 24

| | | | | | | | | | Axitinib |
|---|---|---|---|---|---|---|---|---|---|
| | | Axitinib | HPγCD | Caffeine | HPMC | Benzalkonium | EDTA | Poloxamer | content(μg/ |
| Number | Sample | (mg) | (mg) | (mg) | (mg) | chloride)(mg) | (mg) | 407 (mg) | mL) |
| 1 | HC8A | 6 | 408 | 30 | 5 | — | — | — | 3524.4 |
| | solution | | | | | | | | |

### 2. Method for Determining the Exposure Amount of the Drug

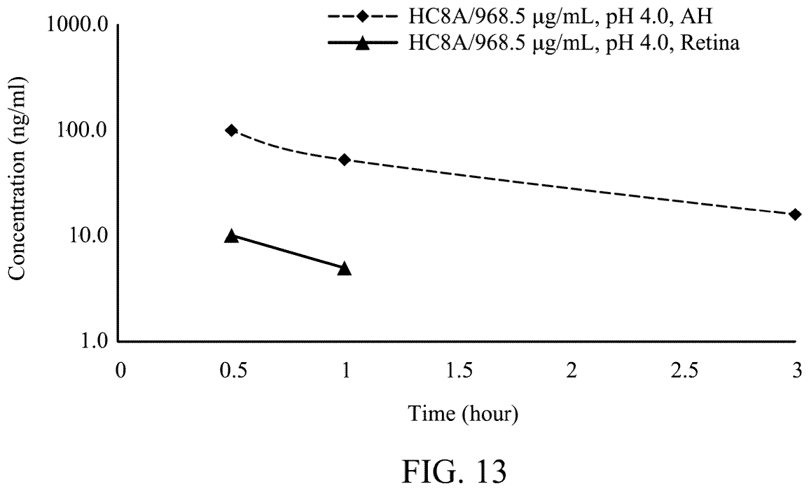

The formulation of HC8A solution of the present disclosure or axitinib-MPP (mucus penetrating particle (MMP) (Kala pharmaceuticals) was respectively administered to the white rabbits according to the method for determining the exposure amount of the drug described in the foregoing Example 4-1, and the results are as shown in Table 25 and FIG. 13.

TABLE 25

| | | | |
|---|---|---|---|
| | Tₘₐₓ | Cₘₐₓ | AUC_{0-3 hour} |
| PK parameter | (Hour) | (ng/mL) | (Hour × ng/mL) |

Aqueous humor

| | | | |
|---|---|---|---|
| HC8A solution, 0.35% | 0.6 ± 0.3 | 244 ± 58 | 377 ± 62 |
| (axitinib concentration: | | | |
| 3.5 mg/mL) | | | |

Retina

| | | | |
|---|---|---|---|
| HC8A solution, 0.35% | 0.5 | 58 ± 10 | 81 ± 22 |
| Axitinib-MPP 2% | 0.5 | 8.39 ± 2.16 | 78.1 ± 5.8 |
| (axitinib concentration: | | | (0-24 hour) |
| 20 mg/mL) | | | |

According to Table 22 and FIG. 13, it can be known that the formulation of the present disclosure can effectively deliver the drug to the posterior chamber, and thus can be effectively applied to the treatment of retro ocular diseases or lesions, such as macular degeneration.

### Example 7-3

### Adjuvant Induced Chronic Uveitis Model (AIT Model)

### 1. Sample Preparation

Samples were prepared according to the formulation as shown in Table 26 below at room temperature. The sample preparation method is analogous to the foregoing Example 1-2. In the sample of this example, Tween 80 is further added to aggregate the sample to form microparticles, and the formed microparticles have an average particle diameter of about 500 nm to 100 μm.

TABLE 26

| | | | | | | | LE |
|---|---|---|---|---|---|---|---|
| | | LE | HPγCD | His | HPMC | Tween 80 | content |
| Number | Sample | (mg) | (mg) | (mg) | (mg) | (mg) | (μg/mL) |
| 1 | HPC8H80LH/TW-PD | 2 | 204 | 80 | 10 | 10 | 1716.8 |

### 2. Experimental Method

Experimental animals: New Zealand White (NZW) rabbits, male, 2-2.5 kg.

Before the start of the experiment, the experimental animals were randomly grouped based on body weight to make each group have similar average body weight and body weight distribution trend.

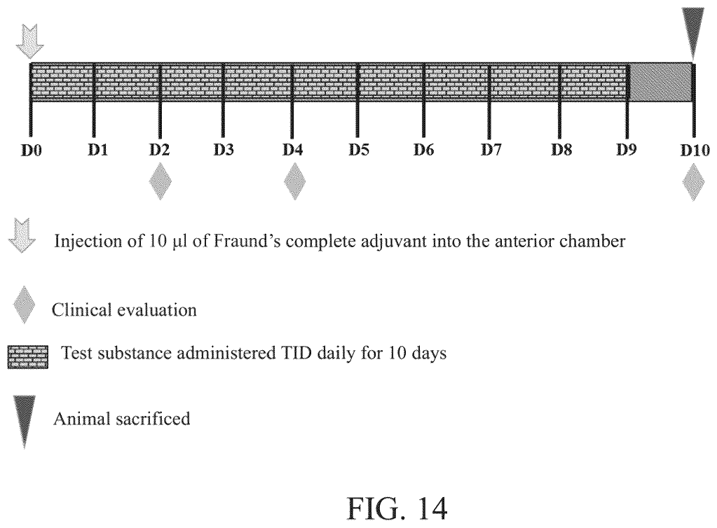

An adjuvant induced chronic uveitis model test was performed according to the time course as shown in FIG. 14.

On Day 0 (DO), the experimental animals were anesthetized with an anesthetic (Zoletil 50:40 mg/kg +xylazine: 10 mg/kg) through intramuscular injection (IM), and then 10 μl Freund's Complete Adjuvant (CFA) was administered to both eyeballs through anterior chamber injection by a 30 G micro syringe.

Thereafter, the test drug was administered on Day 0 (D0). The test substance was administered to both eyes in the form of eye drops, three times a day, each time in a volume of 35 μL/eye, and administered continuously for 10 days.

On Day 2 (D2), Day 4 (D4), and Day 10 (D10), both eyes of the experimental animals were observed with a slit lamp, and the degree of conjunctival congestion, the degree of anterior chamber flare and the condition of uveitis were respectively scored or graded according to the standards of grading as shown in Tables 27, 28, and 29 below to assess the state of intraocular inflammation.

Thereafter, the experimental animals were sacrificed with an excess of CO₂ gas on Day 10 (D10), and the aqueous humor was taken for analyses for infiltration count of inflammatory cell, protein production, and PGE2 production.

TABLE 27
Scoring for conjunctival congestion degree

| Score | Condition |
|---|---|
| 0 | Normal |
| 1 | Mild dilation |
| 2 | Moderate siltation |
| 3 | Diffuse redness |

Bellot J L et al., 1996

TABLE 28
Scoring for anterior chamber flare degree

| Grade | Condition |
|---|---|
| 0 | None |
| 1 | Faint |
| 2 | Moderate (iris and lens details clear) |
| 3 | Marked (iris and lens details hazy) |
| 4 | Intense (fibrin or plastic aqueous) |

THE STANDARDIZATION OF UVEITIS NOMENCLATURE (SUN) WORKING GROUP, 2005

TABLE 29
Scoring for uveitis condition

| | |
|---|---|
| Clinical signs | Grade of uveitis (score) |

Iris hyperemia

| | |
|---|---|
| Absent | 0 |
| Mild | 1 |
| Moderate | 2 |
| Severe | 3 |

Pupil

| | |
|---|---|
| Normal | 0 |
| Miosed | 1 |

Exudate in anterior chamber

| | |
|---|---|
| Absent | 0 |
| Small | 1 |
| Large | 2 |

Hypopyon

| | |
|---|---|
| Absent | 0 |
| Present | 1 |
| Maximum possible score | 7 |

Hoekzema R et al., 1991

### 3. Results

The rabbits were separately administered with vehicle, 0.1% dexamethasone sodium phosphate, and 0.17% HPC8H8OLH/TW-PD (loteprednol etabonate concentration: 1.7 mg/mL) according to the method described above. The results are as shown in FIG. 15 and FIGS. 16A to 1C.

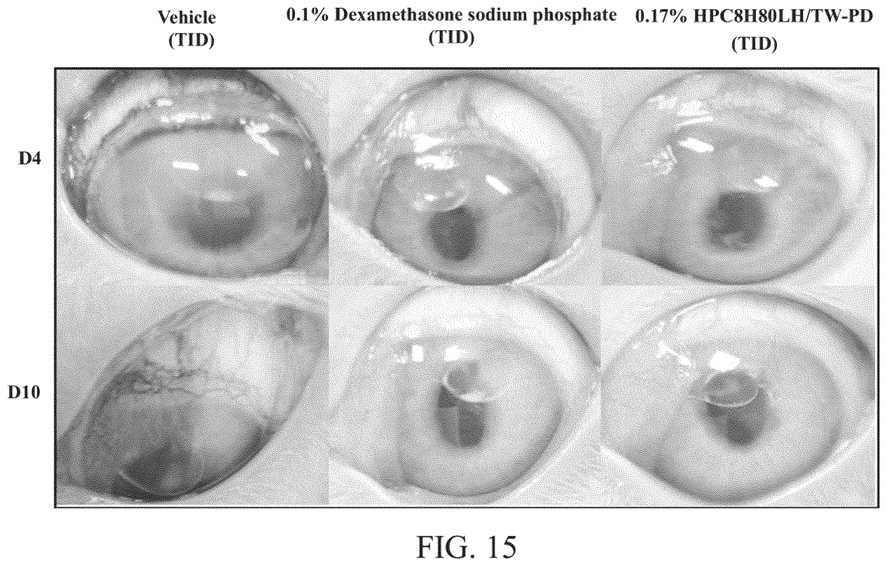

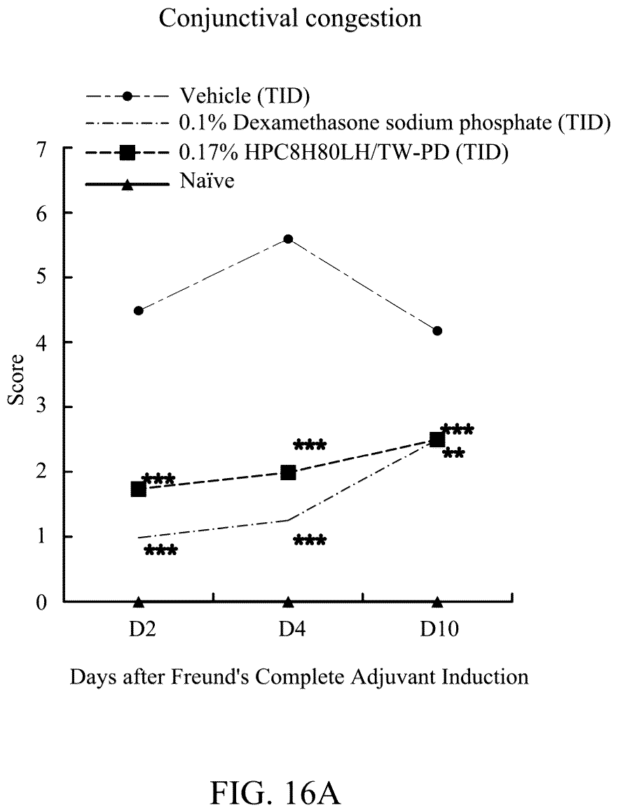

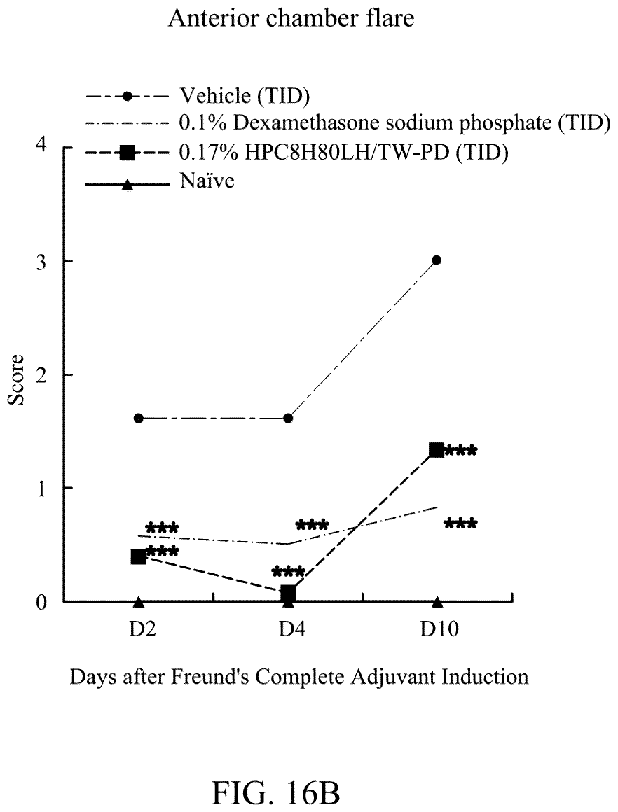

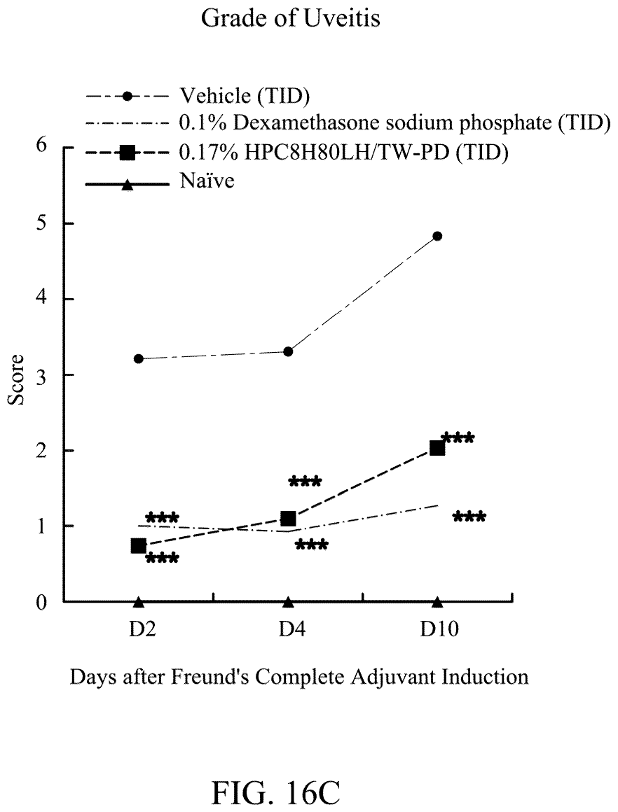

According to FIG. 15 and FIGS. 16A to 16C, it can be known that the formulation of the present disclosure can effectively improve conjunctival congestion, anterior chamber flare and uveitis.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A composition for improving the solubility of poorly soluble substances, comprising:
60-97% by weight of cyclodextrin and/or a derivative thereof;
0.5-4% by weight of at least one water-soluble polymer; and
0.4-30% by weight of at least one water-soluble stabilizer,
wherein the at least one water-soluble stabilizer comprises caffeine, and
wherein the poorly soluble substance is a tyrosine kinase inhibitor.

2. The composition for improving the solubility of poorly soluble substances as claimed in claim 1, wherein the cyclodextrin comprises α-cyclodextrin, β-cyclodextrin, γ-cyclodextrin, δ-cyclodextrin or a combination thereof, and wherein the derivative of cyclodextrin comprises hydroxypropyl modified cyclodextrin, succinyl modified cyclodextrin, methyl modified cyclodextrin or a combination thereof.

3. The composition for improving the solubility of poorly soluble substances as claimed in claim 2, wherein the hydroxypropyl modified cyclodextrin comprises hydroxypropyl-β-cyclodextrin (hydroxypropyl-β-CD) or hydroxypropyl-γ-cyclodextrin (hydroxypropyl-γ-CD).

4. The composition for improving the solubility of poorly soluble substances as claimed in claim 1, wherein the at least one water-soluble polymer comprises hydroxypropyl methyl cellulose (HPMC), hydroxypropyl cellulose, carboxymethyl cellulose (CMC), polyvinylpyrrolidone, (PVP), polyvinyl alcohol, poly(ethylene glycol)-poly(propylene glycol)-poly(ethylene glycol) (PEG-PPG-PEG (ABA)) triblock copolymer or a combination thereof.

5. The composition for improving the solubility of poorly soluble substances as claimed in claim 1, wherein the tyrosine kinase inhibitor is at least one selected from a group consisting of a Type I tyrosine kinase inhibitor, a Type II tyrosine kinase inhibitor, a Type III tyrosine kinase inhibitor, a Type IV tyrosine kinase inhibitor and a Type V tyrosine kinase inhibitor.

6. The composition for improving the solubility of poorly soluble substances as claimed in claim 5, wherein the Type I tyrosine kinase inhibitor comprises cabozantinib, a Type II tyrosine kinase inhibitor comprises axitinib and the Type III tyrosine kinase inhibitor comprises binimetinib.

7. A complex formulation, comprising:
at least one active ingredient wherein the at least one active ingredient is a tyrosine kinase inhibitor; and
a composition for improving the solubility of poorly soluble substances, comprising:
60-97% by weight of cyclodextrin and/or a derivative thereof;
0.5-4% by weight of at least one water-soluble polymer; and
0.4-30% by weight of at least one water-soluble stabilizer, wherein the
at least one water-soluble stabilizer comprises caffeine,
wherein the content of at least one active ingredient in the complex formulation is 0.05-20 wt %.

8. The complex formulation as claimed in claim 7, wherein the tyrosine kinase inhibitor is at least one selected from a group consisting of a Type I tyrosine kinase inhibitor, a Type II tyrosine kinase inhibitor, a Type III tyrosine kinase inhibitor, a Type IV tyrosine kinase inhibitor and a Type V tyrosine kinase inhibitor.

9. The complex formulation as claimed in claim 8, wherein the Type I tyrosine kinase inhibitor comprises cabozantinib, a Type II tyrosine kinase inhibitor comprises axitinib and the Type III tyrosine kinase inhibitor comprises binimetinib.

10. The complex formulation as claimed in claim 7, wherein the cyclodextrin comprises α-cyclodextrin, β-cyclodextrin, γ-cyclodextrin, δ-cyclodextrin or a combination thereof, and wherein the derivative of cyclodextrin comprises hydroxypropyl modified cyclodextrin, succinyl modified cyclodextrin, methyl modified cyclodextrin or a combination thereof.

11. The complex formulation as claimed in claim 10, wherein the hydroxypropyl modified cyclodextrin comprises hydroxypropyl-β-cyclodextrin (hydroxypropyl-β-CD) or hydroxypropyl-γ-cyclodextrin (hydroxypropyl-γ-CD).

12. The complex formulation as claimed in claim 7, wherein the at least one water-soluble polymer comprises hydroxypropyl methyl cellulose (HPMC), hydroxypropyl cellulose, carboxymethyl cellulose (CMC), polyvinylpyrrolidone, (PVP), polyvinyl alcohol, poly(ethylene glycol)-poly(propylene glycol)-poly(ethylene glycol) (PEG-PPG-PEG (ABA)) triblock copolymer or a combination thereof.

13. A liquid formulation of an active ingredient, comprising:
at least one active ingredient, wherein the at least one active ingredient is a tyrosine kinase inhibitor;
a composition for improving the solubility of poorly soluble substances, comprising:
6 60-97% by weight of cyclodextrin and/or a derivative thereof;
7 0.5-4% by weight of at least one water-soluble polymer; and
8 0.4-30% by weight of at least one water-soluble stabilizer, wherein the
at least one water-soluble stabilizer comprises caffeine; and
a solvent,
wherein the content of the at least one active ingredient in the liquid formulation of an active ingredient is 0.01-10% (w/v).

14. The liquid formulation of an active ingredient as claimed in claim 13, wherein the tyrosine kinase inhibitor is at least one selected from a group consisting of a Type I tyrosine kinase inhibitor, a Type II tyrosine kinase inhibitor, a Type III tyrosine kinase inhibitor, a Type IV tyrosine kinase inhibitor and a Type V tyrosine kinase inhibitor.

15. The liquid formulation of an active ingredient as claimed in claim 14, wherein the Type I tyrosine kinase inhibitor comprises cabozantinib, a Type II tyrosine kinase inhibitor comprises axitinib and the Type III tyrosine kinase inhibitor comprises binimetinib.

16. The liquid formulation of an active ingredient as claimed in claim 13, wherein the cyclodextrin comprises a-cyclodextrin, β-cyclodextrin, γ-cyclodextrin, δ-cyclodextrin or a combination thereof, and wherein the derivative of cyclodextrin comprises hydroxypropyl modified cyclodextrin, succinyl modified cyclodextrin, methyl modified cyclodextrin or a combination thereof.

17. The liquid formulation of an active ingredient as claimed in claim 16, wherein the hydroxypropyl modified cyclodextrin comprises hydroxypropyl-β-cyclodextrin (hydroxypropyl-β-CD) or hydroxypropyl-γ-cyclodextrin (hydroxypropyl-γ-CD).

18. The liquid formulation of an active ingredient as claimed in claim 13, wherein the at least one water-soluble polymer comprises hydroxypropyl methyl cellulose (HPMC), hydroxypropyl cellulose, carboxymethyl cellulose (CMC), polyvinylpyrrolidone, (PVP), polyvinyl alcohol, poly(ethylene glycol)-poly(propylene glycol)-poly(ethylene glycol) (PEG-PPG-PEG (ABA)) triblock copolymer or a combination thereof.

19. The liquid formulation of an active ingredient as claimed in claim 13, wherein the liquid formulation of an active ingredient is a liquid dosage form of pharmaceutical formulation.

20. The liquid formulation of an active ingredient as claimed in claim 19, wherein the liquid dosage form of pharmaceutical formulation comprises an oral dosage form, an injection dosage form or an eye drop.
